(19) United States
(12) Patent Application Publication
(10) Pub. No.: US 2005/0182568 A1
(43) Pub. Date: Aug. 18, 2005

(54) Method for evaluating a patients biological condition

(76) Inventors: Christian Duraffourd, Paris (FR); Jean-Claude Lapraz, Paris (FR)

Correspondence Address: OSTROLENK FABER GERB & SOFFEN, 1180 AVENUE OF THE AMERICAS, NEW YORK, NY, 100368403, US

(21) Appl. No.: 10/502,237

(22) PCT Filed: Sep. 11, 2002
(86) PCT No.: PCT/FR02/03089

(30) Foreign Application Priority Data
Sep. 11, 2001 (FR) 0111744

(51) Int. Cl. G06F 19/00; G01N 33/48; G01N 33/50; G01N 15/06; G01N 33/00

(52) U.S. Cl. 702/19; 422/68.1

(57) ABSTRACT

This invention is concerned with a method for evaluating the dynamic biological state of a patient, said method, which involves measuring several elements or substances contained in the blood and interpreting results of the measurements carried out, comprising the following steps (1°) taking a sample of blood from the patient to be examined; (2°) determining hematic substances serving as metabolic and/or tissue parameters; (3°) measuring, on the basis of the determination of step (2°), the totality or part of indexes J1 to J157 defined in the disclosure; and (4°) comparing at least part of said indexes J1 to J157 with similar values obtained at steps (2°) and (3°) on subjects already identified as healthy, to dynamically assess the biological state of the patient to be examined. This invention is also concerned with a software product for executing on a computer steps (3°) and (4°).

### FIELD OF THE INVENTION

This invention is concerned with a new method for evaluating or diagnosing the dynamic biological state of a patient (or a healthy subject), in view to anticipate and to watch its evolution with the passing of time in order to prevent foreseeable abnormalities and/or to correct revealed defects.

### PRIOR ART

Today, every biological method is destined to show the presence or absence of an illness and, if an illness does exist, to specify its nature and degree of gravity. In order to appreciate the optional pathological state of a patient (i) the amount of one or several parameters is determined in a body fluid taken from a patient, for instance blood, plasma, serum, saliva, or urine, then (ii) said amount is compared to the normal value (in general a normal range) which is known from a healthy patient or a pool of healthy patients.

Such a route is substantially of the binary type—yes or no—and static in the sense that the concerned parameter is connected to its sole normal value. That route does not provide any indication regarding the modalities of modifications of said amount and its upholding within the normality range. It allows showing the humeral abnormalities of the patient, but does not allow to appreciate his dynamic biological state and to foresee its evolution with the passing of time. It would surely be more convenient to propose a new technique implying a reading of the ternary or higher multivariable type, in order to make available a dynamic approach for an overall view of evolution.

### AIM OF THE INVENTION

The aim is to provide a novel technical solution allowing to obviate the above cited defects of the prior art. That novel technical solution comprises determining several metabolic and/or tissular parameters, which are known and referenced, measuring multiple indexes from said parameters and comparing said indexes with values obtained from healthy patients or analogous values deduced from medians of international references which are recognized for the different parameters used for subjects who are ideally free of those defects which are looked for.

### SUBJECT OF THE INVENTION

The novel technical solution which is provided according to the invention ensures a method for evaluating (or diagnosing) the dynamic biological state of a patient (either healthy or ill), both on the structure and function points of view, and its evolution with the passing of time in view to correct observed defects, said method, which involves measuring several elements or substances present in blood and interpreting results of performed measures, comprising the following steps:
(1°) providing the blood previously taken from a patient;
(2°) determining in vitro, from said blood, hematic substances as metabolic and/or tissular parameters:
   number of red blood cells (GR),
   number of leukocytes (GB),
   hemoglobin (HG),
   number of neutrophils,
   number of eosinophils,
   number of lymphocytes,
   number of monocytes,
   number of platelets,
   lactate dehydrogenase (LDH),
   creatine phosphokinase (CPK),
   thyroid-stimulating hormone (TSH),
   alkaline phosphatases,
   liver (H1 and H2), bone (O1) and/or intestine (I1, I2 and I3) isoenzymes,
   osteocalcin,
   potassium and calcium, and optionally, at least one of the following substances:
   carcinoembryonic antigen (CEA),
   one or several CA15-3, CA125 and CA19-9 markers,
   acid phosphatases, in particular prostatic acid phosphatase (PAP),
   prostate specific antigen (PSA),
   hourly sedimentation rate (ESR₁),
   bihourly sedimentation rate (ESR₂),
   thyroid hormones, in particular triiodothyronine (FT3) and thyroxine (FT4),
   γ-glutamyl transpeptidases,
   transaminases,
   chlorides and sodium, and
   adrenocorticotropic hormone (ACTH);
(3°) measuring, from step (2°), at least one index selected from the group consisting of following indexes J1-J157:
J1 the so-called genital ratio index, which is the ratio red blood cells/leukocytes,
J2 the so-called genital-thyroid ratio index, which is the ratio neutrophils/lymphocytes,
J3 the so-called adaptation index, which is the ratio eosinophils/monocytes,
J3 being such that J3=eosinophils/monocytes=ACTH/FSH,
J4 the so-called thyroid index, which is the ratio LDH/CPK,
J5 the so-called estrogenic index, which is the ratio TSH/osteocalcin,
J6 the so-called growth index, which is the ratio bone isoenzymes of the alkaline phosphatases/osteocalcin, (O1/osteocalcin),
J7 the so-called turnover index, which is the product TSH×O1
J8 the so-called fibrosis index, J8 being defined by the relation
J8=(TSH)²(osteocalcin)³/100,
J9 the so-called index of thyroid involvement, which is the ratio CA 15-3/CEA,
J10 the so-called index of follicular involvement, which is the ratio CA 125/CEA,
J11 the so-called index of metabolic-hypothalamic involvement, which is the ratio CA19-9/CEA,
J12 the so-called pancreatic index, which is the ratio PAP/PSA,
J13 the so-called global TRH index of adaptation, which is the ratio CA19-9/TSH,
J14 the so-called index of leukocytes mobilization, J14 being defined by the relation J14=(platelets×neutrophils×HG)/(30×leukocytes),
J15 the so-called index of platelets mobilization, J15 being defined by the relation J15=platelets/(60×red blood cells),
J16 the so-called index of thyroid reactivating activity, which is the ratio monocytes/lymphocytes,
J17 the so-called structure/function ratio index, J17 being defined by the relation J17=(neutrophils+basophils+monocytes)/(eosinophils×lymphocytes),
J18 the so-called index of estrogenic fraction # 1, which is the ratio lymphocytes/osteocalcin,
J19 the so-called index of estrogenic fraction #2, which is the ratio neutrophils/monocytes,
J20 the so-called index of metabolic estrogenic fraction, which is the ratio LDH/osteocalcin,
J21 the so-called index of thyroid mobilization of bone metabolism, which is the ratio LDH/bone isoenzymes fraction of the alkaline phosphatases,
J22 the so-called index of thyroid mobilization of bone endocrine metabolism, which is the ratio TSH/bone isoenzymes fraction of the alkaline phosphatases,
J23 the so-called index of relative osteomuscular metabolic activity, which is the ratio CPK/bone isoenzymes fraction of the alkaline phosphatases,
J24 the so-called index of thyroid bone metabolic activity, which is the ratio CPK/osteocalcin,
J25 the so-called catabolism/anabolism ratio index, J25 being the ratio J2/J1,
J26 the so-called index of circulating cortisol, J26 being the ratio J25/J3,
J27 the so-called androgenic index, J27 being the ratio J1/J3,
J28 the so-called adrenal cortex index, J28 being the ratio J26/J27,
J29 the so-called index of adrenal cortex permissiveness, J29 being the ratio J1/J27,
J30 the so-called index of aromatization of estrogens, J30 being the ratio J29/J1,
J31 the so-called level of catabolism, J31 being the ratio J4/J28,
J32 the so-called level of anabolism, J32 being the ratio J31/J25,
J33 the so-called level of metabolic activity efficiency, J33 being defined by the relation J33=(J32+J31)×100/2.25,
J34 the so-called index of bone remodeling, which is the product TSH×J6,
J35 the so-called index of nuclear membrane activity, J35 being the ratio J5/J6,
J36 the so-called adjusted growth index, J36 being the ratio J6/J7,
J37 the so-called anti-growth index, J37 being the ratio 1/J36,
J38 the so-called somatostatin index, J38 being the ratio J37/J26,
J39 the so-called prolactin index, J39 being defined by the relation
J39=(J38/J6)×TSH,
J40 the so-called level of membrane expansion, J40 being the product J31×J36,
J41 the so-called level of structural expansion, J41 being the product J32×J35,
J42 the so-called apoptosis index, J42 being the ratio J41/J40,
J43 the so-called adjusted apoptosis index, J43 being the ratio J42/J35,
J44 the so-called level of membrane fracture, J44 being defined by the relation J44=J33/(TSH×J7),
J45 the so-called necrosis index, J45 being the ratio J44/J42,
J46 the so-called level of activity of total androgens, J46 being the product J5×J1
J47 the so-called rate of adrenal cortex androgens, J47 being defined by the relation J47=J46/(1+J27),
J48 the so-called rate of genital androgens, J48 being defined by the relation J48=(J46−J47),
J49 the so-called progesterone index, J49 being defined by the relation
J49=J5/(J48×J3),
J50 the so-called level of activity of genital estrogens, J50 being defined by the relation J50=J5/(1+J30),
J51 the so-called rate of aromatized estrogens, J51 being defined by the relation J51=J5−J50,
JJ53 the so-called folliculin index, J53 being defined by the relation J53=20×(J5/J49),
J54 the so-called insulin index, J54 being defined by the relation J54=(100×J25)/(J7×TSH),
J55 the so-called demyelinization index, J55 being defined by the relation J55−J541(J36×J6),
J56 the so-called index of DNA fracture, J56 being defined by the relation J56=(100×J5×J6×J41)/(J7×J35×J42×J45),
J57 the so-called index of nucleocytoplasmic pathogenicity, J57 being defined by the relation J57=(1.7×J56)/J44,
J58 the so-called index of cellular fracture, J58 being defined by the relation J58=2.5×J44×J56/J45,
J59 the so-called index of carcinogenesis, J59 is the ratio J57/J42,
J60 the so-called index of comparative carcinogenesis, J60 being defined by the relation J60=(10×J58)/J43,
J61 the so-called index of active cellular permeability, J61 being defined by the relation J61=J6×J34/J54,
J62 the so-called index of adjusted active cellular permeability, J62 being defined by the relation J62=(J61×J29)/J26,
J63 the so-called index of passive cellular permeability, J63 being defined by the relation J63=J45×J35×J68×10 (wherein J68 is defined as indicated below),
J64 the so-called index of active intracellular osmolar gradient, J64 being defined by the relation J64=100×J54×J40×J35/J3,
J65 the so-called index of adjusted active intracellular osmolar gradient, J65 being defined by the relation J65=(J64×J29)/J26,
J66 the so-called index of passive intracellular osmolar gradient, J66 being defined by the relation J66=(10×J43×J53)/(J45×J8),
J67 the so-called oxidation-reduction index, J67 being defined by the relation
J67=(100×J45×J40×J41×J54)/(J71×J8×J38), (wherein J71 is defined as indicated below),
J68 the so-called index of corticoadrenal adaptation/permissiveness, J68 being defined by the relation J68=J26−J29−J28,
J69 the so-called adaptogenic index which is the ratio K/Ca,
J70 the so-called βMSH/αMSH index, (differential melanocyte-stimulating hormones), J70 being the ratio J4/J69,
J71 the so-called apoptosis bis index, J71 being defined by the relation
J71=J35/(J36×J25),
J72 the so-called amylosis index, J72 being defined by the relation
J72=(J38×J53×J55×TSH)/(J4×J5×J54),
J73 the so-called index of amylosis risk, J73 being the ratio J8/J67,
J74 the so-called index of insulin resistance, J74 being the ratio J38/J54,
J75 the so-called upstream index # 1, J75 being the ratio J4/J9,
J76 the so-called upstream index #2, J76 being the ratio J4/J10,
J77 the so-called upstream index #3, J77 being the ratio J4/J11,
J78 the so-called global upstream index #1, J78 being the ratio J75/J76,
J79 the so-called global upstream index #2, J79 being the ratio J75/J77,
J80 the so-called global upstream index #3, J80 being the ratio J76/J77,
J81 the so-called index of thyroid output #1, J81 being the ratio J4/TSH,
J82 the so-called index of free radicals, J82 being the ratio J67/J54,
J83 the so-called adjusted index of free radicals, J83 being defined by the relation J83=(J67+J64)/(J54+J74),
J84 the so-called comparative index of free radicals, J84 being defined by the relation J84=(J67+(100×J40)/(J54+J74),
J85 the so-called index of free radical nocivity, J85 being defined by the relation J85=((J82+J83+J84)×J56)/(3×J71),
J86 the so-called adjusted apoptosis index (B), J86 being the ratio J71/J35,
J87 the so-called index of active histamine, J87 being defined by the relation
J87=(eosinophils×platelets×J3)/J52,
J88 the so-called index of potential histamine, J88 being defined by the relation J88=(J87×J63)/(potassium×J70)
J89 the so-called TRH index, J89 being the ratio TSH/FT4, J90 the so-called index of relative intrathyroid TRH activity, which is the ratio FT3/FT4,
J91 the so-called index of carcinogenic expansion, J91 being the ratio J60/J59,
J92 the so-called index of cancer potential, J92 being the product J91×J54×J85,
J93 the so-called adenosis index, J93 being the ratio J8/J91,
J94 the so-called ischemia reperfusion index, J94 being defined by the relation J94=10×J34×J43/J33,
J95 the so-called thrombogenic index, J95 being defined by the relation J95=10×J34×J42×J45/J33,
J96 the so-called thrombotic index, J96 being defined by the relation J96=J95×J87×J1/10,
J97 the so-called adjusted genital ratio index, J97 being defined by the relation J97=(J14×Red cells)/(Leukocytes×J15)=J14×J1/J15,
J98 the so-called musculotropic index, J98 being defined by the relation J98=J97×(CPK/O1),
J99 the so-called adjusted estrogenic index, J99 being defined by the relation J99=(J5×(osteocalcin+1)/(osteocalcin+1−J98)),
J100 the so-called genital androgeny index, J100 being defined by the relation J100=(J98/J81)×J99×(J97)²/(J3+J97),
J101 the so-called comparative genital androgeny index, J101 being defined by the relation J101=(2×(TSH)²×CPK)/(J4×osteocalcin×01),
J102 the so-called “starter” index, J102 being the ratio J14/J15,
J103 the so-called adjusted index of thyroid reactivating activity, J103 being the product J16×J2,
J104 the so-called pro-inflammatory index, J104 being the product J103×J69,
J105 the so-called index of inflammation, J105 being the product J104×J45,
J106 the so-called comparative index of inflammation, J106 being defined by the relation J106=J105/(((ESR₂/2)+ESR₁)/2)/ESR₁,
J107 the so-called interleukin 1 index, J107 being defined by the relation J107=(J16×J38)/(J103×J37),
J108 the so-called DHEA index, J108 being defined by the relation J108=(J29×J30×J47×J51×J98×1000)/(J49×J27×J100),
J109 the so-called serotonin index, J109 being defined by the relation J109=(10×J102)/(J54×J74),
J110 the so-called adjusted demyelinization index, J110 being the product J55×J102,
J111 the so-called expansiveness index #1, J111 being the ratio J36/J35, J112 the so-called expansiveness index #2, J112 being the ratio J40/J41,
J113 the so-called global expansiveness index, J113 being defined by the relation J113=(J111×J112)/J45,
J114 the so-called ACTH index, J114 being the ratio J108/J26,
J115 the so-called PTH index, J115 being defined by the relation J115=(calcium×osteocalcin×TSH)/J4,
J116 the so-called index of gonadotropic output, J116 being defined by the relation J116=1/(J×J53),
J117 the so-called index of pelvic congestion, J117 being defined by the relation J117=(J59/J60)×(J94/J33),
J118 the so-called index of splanchnic congestion, J118 being the ratio J117/J14,
J119 the so-called growth score index, J119 being defined by the relation J119=(J6×J37)/(J36×J38),
J120 the so-called GH growth score index, J120 being defined by the relation J120=(J6×J37)/J36,
J121 the so-called TRH/TSH ratio index, J121 being the ratio J72/J93,
J122 the so-called index of thyroid efficiency, J122 being the ratio J4/J2,
J123 the so-called index of relative thyroid efficiency, J123 being the ratio J122/J81,
J124 the so-called index of oxidation, J124 being defined by the relation J124=(100×J36×J54×J122)/(J74×J26),
J125 the so-called index of reduction, J125 being the ratio J124/J67,
J126 the so-called pro-amyloid index, J126 being the product J125×J74,
J127 the so-called index of amyloid risk, J127 being the ratio J8/J124,
J128 the so-called index of thyroid output #2, J128 being the product J2×J4,
J129 the so-called comparative index of thyroid output, J129 being the ratio J128/J81,
J130 the so-called index of estrogenic fraction #3, J130 being the ratio 1/J1,
J131 the so-called index of estrogenic fraction #4, J131 being the product J18×J19,
J132 the so-called index of estrogenic fraction #5, J132 being the product J19×J130,
J133 the so-called general index of estrogenic fraction, J133 being the product J18×J19×J130,
J134 the so-called index of estrogenic fraction #6, J134 being defined by the relation J134=1/(osteocalcin×J2),
J135 the so-called index of estrogenic fraction #7, J135 being the product J18×J19×J134,
J136 the so-called index of estrogenic fraction #8, J136 being the ratio J2/osteocalcin,
J137 the so-called general quantitative estrogenic index, J137 being the product (J18+J19)×(leukocytes/100),
J138 the so-called index of specific estrogenic fraction, J138 being the product J5×(J98+1),
J139 the so-called comparative estrogenic index #1, J139 being the ratio J133/(J5×100),
J140 the so-called comparative estrogenic index #2, J140 being the ratio J133/(J99×100),
J141 the so-called global comparative estrogenic index, J141 being the ratio J133/(J5×J99×100),
J142 the so-called index of somatotropic estrogenic output, J142 being the ratio J133/J144 (where J144 is defined as indicated below),
J143 the so-called index of quantitative organotissular estrogenic output, J143 being the ratio J137/J144 (where J144 is defined below),
J144 the so-called FSH index #1, J144 being the ratio J114/J3,
J145 the so-called LH index #1, J145 being the product J114×J27,
J146 the so-called FSH index #2, J146 being the ratio J145/J1,
J147 the so-called LH index #2, J147 being the product J144×J1,
J148 the so-called index of progesterone output, J148 being the ratio J49/J138,
J149 the so-called ketonic index, J149 being the ratio J102/J54,
J150 the so-called index of total subliminal TRH, J150 being the product TSH×(CA19-9)×J90,
J151 the so-called index of active carcinogenesis, J151 being the product J59×J113,
J152 the so-called comparative index of active carcinogenesis, J152 being the product J60×J113,
J153 the so-called gonadothyrotropic index, J153 being the ratio TSH/J2,
J154 the so-called index of global tissular estrogenic fraction, J154 being the ratio J140/J139,
J155 the so-called index of muscle destruction, J155 being the ratio J36/J101,
J156 the so-called amyloid score index, J156 being defined by the relation J156=(J2×J53×J72×J94×J110×J126×J127)/(J4×J5×J67×J19×J20),
J157 the so-called adjusted necrosis index, J157 being the product LDH×J45; and,
(4°) comparing at least one of the J1-J157 indexes with the corresponding result obtained according to steps (2°) and (3°) with human beings already recognized as being healthy, in order to appreciate dynamically the biological state of the patient to be tested.

According to a further aspect of the invention, a software product is provided, which allows to carry out steps (3°) and (4°) of said method. Said software product, which is (directly or indirectly loadable) in the (internal or auxiliary) memory of a digital or analog computer, comprises portions of computer code to perform steps (3°) and (4°) of the method regarding the evaluation of the dynamic biological state of a patient.

### DETAILED DESCRIPTION OF THE INVENTION

The method according to this invention provides a value, which is (i) dynamic vis-à-vis the static image given by the binary frame of the prior art teaching, (ii) diagnostic with respect to the true biological etiology of each patient vis-à-vis the specific etiology of the illness, and (iii) predictive which is highly more precise both in the pathogenic risk and the pathological future.

Carrying out the present method requires a minimal number of indexes exploring the functions involved in the symptomatology and/or pathology of the patient to be tested. To that aim it is interesting to determine in step (2°) one or several hematic substances among those that follow:
(a) those intervening as essential parameters:
number of red blood cells (GR),
number of leukocytes (GB),
hemoglobin (HG),
number of neutrophils,
number of eosinophils,
number of lymphocytes,
number of monocytes,
number of platelets,
lactate dehydrogenase (LDH),
creatine phosphokinase (CPK),
thyroid-stimulating hormone (TSH),
alkaline phosphatases,
liver (H1 and H2), bone (O1) and/or intestine (I1, I2 and I3) isoenzymes,
osteocalcin,
potassium and calcium;
(b) those intervening as specific parameters vis-à-vis certain pathologies:
   carcinoembryonic antigen (CEA),
   one or several CA15-3, CA125 and CA19-9 markers,
   acid phosphatases, in particular prostatic acid phosphatase (PAP),
   prostate specific antigen (PSA);
(c) those intervening as supplemental parameters:
   hourly sedimentation rate (ESR₁),
   bihourly sedimentation rate (ESR₂),
   thyroid hormones, in particular triiodothyronine (FT3) and thyroxine (FT4); and,
(d) those useful for orienting the choice of an index or a group of indexes:
   γ-glutamyl transpeptidases,
   transaminases,
   chlorides and sodium, and
   adrenocorticotropic hormone (ACTH).

In practice, parameters belonging to the above groups (a), (b) and (c) are determined in vitro. Advantageously, are determined:
the totality of parameters (a),
the totality or a portion of the parameters (b), and
at least one of the parameters (c) or their totality, in view of the symtomatology and/or pathology of the human being to be tested.

In step (3°) of the method according to the invention, it is sufficient in some cases to measure a single index among the J1-J157 indexes, see for instance Tables XVIII (J70), XIX (J8), XXI (J74), XXI (J54) and XXV (J42). In some other cases, it is sufficient to measure only two indexes among the J1-J157 indexes, see for instance Tables XX (J42 and J43), XXIII (J92 and J93) and XXIV (J26 and J28).

In practice, in step (3°) it is recommended to measure at least 8 indexes J, advantageously at least 10 indexes J and preferably at least 15 indexes J among the J1-J157 indexes.

According to a particular mode for carrying out the method of the invention, it is recommended to measure at least 8 indexes J, advantageously at least 10 indexes J and preferably at least 15 indexes J among the J1-J24 indexes.

As a variant, at least one portion of the J1-J24 indexes can be replaced by scores of functions involved in the symptomatology and/or pathology of the human being to be studied, thoses scores being illustrated by an index from J25 to J157 or by one or several groups of indexes among the J25-J157 indexes. Those scores can be used in diagnosing illness or established incidents, during establishment or even evaluation of potential risk at every stage (in French: “risque encouru à tous termes”).

Said scores of functions involved in the symptomatology and/or pathology are for instance, but by no way limitative, scores of pregnancy, menstruation, cardiovascularity, thrombosis, Alzheimer's disease, atherosclerosis, cancer, sudden death risk. The sudden death risk comprises in particular infarct risk, aneurism risk, anaphylactia risk. Cancer risk comprises the one of disease in a general manner, as well as the one of its localization, in particular brain, colon, prostate gland and breast cancers.

The indexes concerned with the score of menstruation are indicated in Example 1 hereinafter. Thoses related to the score of ovulation are given in Example 2 below.

Where other health conditions and/or syndromes are concerned, the indexes to be analyzed according to the invention are the following ones:
for somatotropic condition (growth and antigrowth): J6, J8, J36, J37, J38, J39, J54, J67, J74, J82, J85, J319 and J120;
for thyroid condition: J2, J4, J16, J39, J68, J72, J74, J81, J93, J103, J121, J128 and J129;
for adaptation syndrome and suprarenal condition: J3, J14, J15, J26, J27, J28, J29, J34, J39, J68, J69, J70, J87, J88, J94, J95, J96 and J102;
for nuclear growth (to be used in case of carcinogenesis): J1, J5, J30, J35, J41, J42, J43, J45, J46, J47, J48, J49, J50, J51, J53, J56, J57, J59, J60, J91, J92, J97, J101 and J141;
for general metabolic activity condition: J7, J8, J25, J33, J34, J40, J44, J45, J61 and J98; and,
for rhabdomyolisis risk (i.e. risk of muscular wasting): J22, J34, J54, J95, J98 and J155.

As a further variant, in step (4°), the comparison of the obtained indexes with those resulting from median values of parameters for human beings recognized as healthy, who are in particular at an ideal equilibrium state. Those median values are advantageously determined on a pool of healthy (male or female) subjects according to steps (2°) and (3°) of the method of this invention. In some cases said median values are depending upon gender.

For working this invention, five levels of function measures are distinguished:
Level 1: functional state detection,
Level 2: complete functional state,
Level 3: specialized functional conditions (for instance liver, hypophysis or thyroid, as well as hydratation, muscular activity),
Level 4: examinations per illnesses or syndromes (in particular thrombosis, Alzheimer's disease, multiple sclerosis, menopause, osteoporosis, ageing, cardiovascular diseases, effort (sport, army), psychiatric diseases, cancer, cholesterol),
Level 5: indexes of specialists (in particular neurology, cardiology, cancerology).

The method provided by this invention allows diagnosing and evaluating the dynamic biological state of a patient (or a healthy subject) from the structure point of view, from the reciprocal interactions (or functionings) point of view, and from the point of view of their evolutions with the passing of time, with the aim of (i) correcting established defects, (ii) possibly preventing foreseeable defects, with respect to the biological state which has been determined in its elements and their reciprocal interactions, from cell to organic level.

In practice, one looks for at least one abnormality at the level of at least one index J (in particular among J1-J24 or J1-J157), then depending on this abnormality one studies the indexes which are linked to or implied by the above cited symptomatology, pathology and/or scores. After that, from the results thus obtained, one determines what should be done to cure this abnormality or to prevent risks, illnesses or pathologies linked to or resulting from the foreseeable evolution of the patient state.

Each index J of the invention is used to quantify a function or an interaction. The indexes concerned with a same pathology or score allow evaluating in fine the efficiency or effectiveness of a function vis-à-vis the metabolic or tissular needs required by the organism. With all the concerned indexes, the practioner has a precise image of the functionality, system by system, and of the real level of the activity of each system.

Thus the method according to the invention provides a specific study of the activity level of each function separately or taken in their global or punctual, general or local interactions. Said method consequently allows to determine or to appreciate pathological tendencies of the organism, the evolution of a given pathology with the passing of time and the degree of said pathology.

Therefore, said method gives an evaluation of the dynamic biological state of a patient to be tested and provides a profitable help in diagnosing.

In step (1°), a blood sample taken from the patient to be tested is used. Optionally, said blood sample can be added with an anticoagulant agent, such as (preferably) heparin or (possibly) hirudin.

In step (2°), parameters are measured according to classical techniques, and the results thus obtained are given in units or subunits, which are usual in the art. If the same units were not used for given parameters, the values of indexes J, resulting from those parameters in step (3°), could not be compared in step (4°).

Enzymes are measured according to techniques of biological activity known per se and the results are expressed in international units (IU), in particular as IU/L or IU/mL:
LDH and CPK are measured according to their global activity and, optionally, can be isolated according to a method known per se (for instance by electrophoresis);
alkaline phosphatases are also measured according to their global activity and after can be isolated according to a method known per se for performing supplemental researches;
transaminases are measured according to their specific activities (in particular their hepatic and muscular ones);
γ-glutamyl transpeptidases are measured according to their global activity.

Among the enzymes, the PAP ones are the sole to be expressed in a weight/volume ratio, namely in ng/L.

Oligoelemnts (K, Na and Cl) are measured according to common techniques of dosage, and the results are expressed in milliequivalent/L.

Osteocalcin is generally determined according to a RIA method and the results are expressed in ng/L.

Hemoglobin is measured according to common techniques and the results are expressed in g/L.

Red blood cells numeration is performed by direct or automated counting and the results are given in thousands/mL.

Leukocytes numeration is performed by direct or automated counting and the results are given in real number/mL.

Platelets numeration is performed by direct or automated counting and the results are given in thousands/mL.

Leukocytes formula is made by direct or automated counting and the results are given in a percentage of the total number of leukocytes.

Sedimentation rate is made by direct or automated measure and the results are expressed in hourly (ESR₁) or bihourly (ESR₂) mm.

Thyroid-stimulating hormone (TSH) is measured by a RIA method and the results are expressed in μIU/L.

Total cholesterol and HLD-cholesterol are measured according to common technique of dosage and the results are expressed in g/L.

Tissue markers, used as tissular parameters are determined according to a technique known per se (in particular by RIA) and the results are expressed in ng/L.

Thyroid hormones (FT3 and FT4) are measured by a RIA or EIA technique and the results are expressed in ng/L.

For information, here is the signification of the indexes of this invention.
J1 A/O the so-called genital ratio index expresses tissular androgenic activity relative to estrogenic activity,
J2 the so-called genital-thyroid ratio index (G/T) expresses gonadic activity relative to thyroid activity; in addition, when it increases it bears witness to an efficient thyroid activity, and when it decreases to a rising TSH activity, whatever the absolute level of the thyroid activity,
J3 the so-called adaptation index expresses ACTH activity in its adaptative function relative to FSH activity,
J4 the so-called thyroid index expresses the metabolic activity of the thyroid,
J5 the so-called estrogenic index expresses estrogenic endocrinometabolic activity,
J6 the so-called growth index expresses growth hormone metabolic activity,
J7 the so-called “turnover” index expresses the speed of tissular renewal: when it increases it shows a slowdown of this renewal, conversely when it decreases it shows its acceleration,
J8 the fibrosis index expresses the fibrotic activity of the organism; this activity ranges from the mere isolation of a tissue or organ to the sclerotic degeneration of an organic or tissular group,
J9 the index of thyroid involvement expresses the share of the relative responsibility of the thyrotropic axis in the pathogenicity of the organism,
J10 the so-called index of follicular involvement expresses the share of the relative responsibility of the gonadotropic axis in the pathogenicity of the organism,
J11 the so-called index of metabolic-hypothalamic involvement expresses the share of the relative metabolic responsibility of the thyrosomatotropic axis in the so-called pathogenicity of the organism,
J12 the so-called pancreatic index expresses the metabolic activity of tissular nutrition by the exocrine pancreas within the somatotropic axis,
J13 the so-called global TRH index of adaptation expresses the metabolic activity of TRH relative to that aspect of its endocrine activity concerned with the thyrosomatotropic adaptation function,
J14 the so-called index of leukocytes mobilization expresses the adaptative releasing capacity of the reserve leukocytes withheld within the splanchnic area on the hepatic side,
J15 the so-called index of platelets mobilization expresses the adaptative releasing capacity of the reserve platelets withheld within the splanchnic area on the splenic side,
J16 the so-called index of thyroid reactivating activity expresses the level of stimulation of the thyrotropic axis by the locus ceruleus; by extension, it bears witness to the degree of maladaptation of the organism,
J17 the so-called structure: function ratio index expresses the adaptative stimulation of structural tissular mechanisms relative to that of functional tissular mechanisms,
J18 the so-called index of estrogenic fraction #1 expresses the share of the relative estrogenic endocrinometabolic adaptative activity,
J19 the so-called index of estrogenic fraction #2 expresses the liminal adaptative share of the tissular activity of estrogens,
J20 the so-called index of metabolic estrogenic fraction expresses the relative share of estrogenic metabolic activity,
J21 the so-called index of thyroid mobilization of bone metabolism expresses the effective relative share of thyroid osteoclastic activity,
J22 the so-called index of thyroid mobilization of bone endocrine metabolism expresses the relative endocrinometabolic share of thyroid osteoclastic activity,
J23 the so-called index of relative osteomuscular metabolic activity expresses the share of the metabolic activity of muscle relative to that of bone,
J24 the so-called index of thyroid bone metabolic activity expresses the relative share of thyroid osteoblastic activity,
J25 the so-called catabolism/anabolism ratio index expresses the catabolic activity of the organism relative to its anabolic activity,
J26 the so-called index of circulating cortisol expresses the secretory activity of cortisol by the adrenal cortex and its excretion during the adaptation syndromes,
J27 the so-called androgenic index expresses the activity of androgens of gonadic origin relative to that of androgens of adrenal origin,
J28 the so-called adrenal cortex index expresses the level of the global endocrine activity of the adrenal cortex,
J29 the so-called index of adrenal cortex permissiveness expresses the level of permissive activity exerted by the adrenal cortex on the secretory activity of the other endocrine glands,
J30 the so-called index of aromatization of estrogens expresses the share of the aromatizing activity of the adrenal cortex relative to its other activities,
J31 the so-called level of catabolism expresses the level of the catabolic activity of the organism,
J32 the so-called level of anabolism expresses the level of the anabolic activity of the organism,
J33 the so-called level of metabolic activity efficiency expresses the level of the general efficiency of the organism with regard to production as well as to distribution,
J34 the so-called index of bone remodeling expresses the level of bone remodeling and the extent of bone impairment; it also bears witness to the general level of the metabolism, and specifically to its adaptation activity,
J35 the so-called index of nuclear membrane activity expresses the level of the metabolic activity of the nucleus relative to that of the membrane; it also reflects their degree of structural dependence relative to their degree of adaptative functional dissociation,
J36 the so-called adjusted growth index expresses the level of intracellular activity of growth factors,
J37 the so-called anti-growth index expresses the global level of activity of anti-growth factors as a whole,
J38 the so-called somatostatin index expresses the level of activity of somatostatin; it reflects indirectly the relative level of activity of the exocrine pancreas,
J39 the so-called prolactin index expresses the level of activity of prolactin;
   it bears witness to the level of stimulation of the general adaptation syndrome and of its systematized units,
J40 the so-called level of membrane expansion expresses the level of metabolic activity of the membrane; it allows its quantitative evaluation relative to the general structural metabolic activity,
J41 the so-called level of structural expansion expresses the level of metabolic activity of the nucleus; it allows its quantitative evaluation relative to the general structural metabolic activity,
J42 the so-called apoptosis index expresses the level of apoptosic activity of the organism as a whole,
J43 the so-called adjusted apoptosis index expresses the degree of physiological apoptosic activity of the organism as a whole relative to apoptosic activity resulting from dysfunctional adaptation,
J44 the so-called level of membrane fracture expresses the degree of fragility of the membranes—their risk of rupture,
J45 the so-called necrosis index expresses the level of cellular explosion related to a necrotic phenomenon relative to cellular destruction caused by apoptosis,
J46 the so-called level of activity of total androgens expresses the level of activity of the androgens taken as a whole,
J47 the so-called rate of adrenal cortex androgens expresses the level of activity of androgens originating from the adrenal cortex,
J48 the so-called rate of genital androgens expresses the level of activity of androgens of gonadic origin,
J49 the so-called progesterone index expresses the level of activity of progesterone,
J50 the so-called level of activity of genital estrogens expresses the level of activity of estrogens of gonadic origin,
J51 the so-called rate of aromatized estrogens expresses the relative share of compensatory estrogens obtained through the aromatization of androgens,
J52 the so-called adrenal cortex index expresses the level of the global endocrine activity of the adrenal cortex,
J53 the so-called folliculin index expresses the level of endocrine activity of the gonadotropic axis in its specific folliculin fraction,
J54 the so-called insulin index expresses the level of functional endocrinometabolic activity of insulin,
J55 the so-called demyelinization index expresses the relative functional adaptative activity of insulin in its chronological relationship with growth hormone activity,
J56 the so-called index of DNA fracture expresses the degree of fragility of the nuclei—their risk of rupture,
J57 the so-called index of nucleocytoplasmic pathogenicity expresses the level of pathogenicity of the nucleus through the metabolic dysregulation that it induces within the cytoplasm, which is exacerbated by the solidity of the membrane,
J58 the so-called index of cellular fracture expresses the global degree of fragility of the cells—their risk of rupture,
J59 the so-called index of carcinogenesis expresses the potential level of carcinogenesis of the organism through its nucleocytoplasmic instability in relation to its inadequacy in apoptosis,
J60 the so-called index of comparative carcinogenesis expresses the level of functional carcinogenesis relative to the level of pathological apoptosis,
J61 the so-called index of active cellular permeability expresses the degree of dynamic trans-membrane permeability,
J62 the so-called index of adjusted active cellular permeability expresses the degree of dynamic structural trans-membrane permeability relative to functional permeability,
J63 the so-called index of passive cellular permeability expresses the degree of strictly osmotic trans-membrane permeability,
J64 the so-called index of active intracellular osmolar gradient expresses the level of maintenance of intracellular osmolarity,
J65 the so-called index of adjusted active intracellular osmolar gradient expresses the level of maintenance of structural intracellular osmolarity relative to functional intracellular osmolarity,
J66 the so-called index of passive intracellular osmolar gradient expresses the relative level of intracellular osmolarity linked to strictly osmotic trans-membrane permeability,
J67 the so-called oxidation-reduction index expresses the final level of the organism's redox activity: that is, after the oxidative activity and the reductive reaction have taken place,
J68 the so-called index of corticoadrenal adaptation/permissiveness expresses the level of the adaptative activity of the adrenal cortex relative to its permissive activity,
J69 the so-called adaptogenic index expresses the relative level of pineal gland participation in the non-circular reactivating of adaptation,
J70 the so-called βMSH/αMSH index (differential hormones stimulating the melanocytes) expresses the relative contribution to the stimulation of an integrated adaptation by, respectively, the programmed loop (long loop—pituitary/periphery) and the intercurrent added loops (short epiphyseal loop),
J71 the so-called apoptosis index (B) expresses the level of apoptosis of the organism as a whole,
J72 the so-called amylosis index expresses the level of sollicitation of the amyloid activity of the organism—an activity of structural protection calling for a ‘bridging’ energy in the face of a functional over-demand for immediate energy (internal desynchronization of the somatotropic axis),
J73 the so-called index of amylosis risk expresses the organism's relative risk of amyloid degeneration in its present functional equilibrium,
J74 the so-called index of insulin resistance expresses the relative level of functional inhibition of the membrane activity of insulin, independent of temporary inhibition linked to the general adaptation syndrome,
J75 the so-called upstream index #1 expresses the relative part played by the thyroid, within the thyrotropic axis, in the genesis and maintenance of the current pathology; by extension, it helps one to evaluate the role of peripheral as compared to central endocrine activity in the genesis and maintenance of the current pathology,
J76 the so-called upstream index #2 expresses the relative part played by the thyroid, within the metabolic equilibrium resulting from the activity of the follicular fraction of the gonadotropic axis, in the genesis and maintenance of the current pathology; by extension, it helps one to evaluate the role of peripheral endocrinometabolic activity as compared to axial centripetal endocrine activity in the genesis and maintenance of the current pathology,
J77 the so-called upstream index #3 expresses the relative metabolic part played by the thyroid, through the compensatory activity of the thyrosomatotropic axis, in the genesis and maintenance of the current pathology; by extension, it helps one to evaluate the role of peripheral metabolic as compared to central metabolic activity in the genesis and maintenance of the current pathology,
J78 the so-called global upstream index #1 expresses the part played by the follicular fraction of the gonadotropic axis relative to the part played by the thyrometabolic fraction of the thyrotropic axis in the genesis and maintenance of the current pathology; by extension, it helps one to evaluate the role of centripetal axial endocrine activity as compared to central endocrine activity,
J79 the so-called global upstream index #2 expresses the part played by the compensatory activity of the thyrosomatotropic axis relative to the activity of the thyrometabolic fraction of the thyrotropic axis in the genesis and maintenance of the current pathology; by extension, it helps one to evaluate the role of central metabolic as compared to central endocrine activity,
J80 the so-called global upstream index #3 expresses the share of the compensatory activity of the thyrosomatotropic axis relative to the activity of the follicular fraction of the gonadotropic axis; by extension, it helps one to evaluate the role of central metabolic activity as compared to centripetal axial endocrine activity,
J81 the so-called index of thyroid output #1 expresses thyroid metabolic activity relative to the level of hypophyseal stimulation; by extension, it helps one to evaluate the threshold of the thyroid's response when stimulated by the pituitary gland,
J82 the so-called index of free radicals expresses the global circulating rate of free radicals,
J83 the so-called adjusted index of free radicals expresses the residual circulating rate of free radicals,
J84 the so-called comparative index of free radicals expresses the rate of the free radicals with a structural purpose relative to the rate of the free radicals with a functional purpose,
J85 the so-called index of free radical nocivity expresses the rate of toxic free radicals relative to the rate of biologically justified free radicals,
J86 the so-called adjusted apoptosis index (B) expresses the rate of anticipated apoptosis relative to the physiological rate of apoptosis,
J87 the so-called index of active histamine expresses the level of active histamine in circulation,
J88 the so-called index of potential histamine expresses the level of histamine carriers, both fixed and mobile,
J89 the so-called TRH index expresses the relative share of TRH's vertical activity within the thyrotropic axis,
J90 the so-called index of relative intrathyroid TRH activity expresses the relative role of TRH in the endocrine activity of the thyroid,
J91 the so-called index of carcinogenic expansion expresses the degree and rapidity of anarchic tissue development within the organism; by extension, it allows one to track the progression of a known tumor, the degree and speed of its development,
J92 the so-called index of cancer potential expresses the relative level of the endocrinometabolic factors favourable to dysplasia,
J93 the so-called adenosis index expresses the relative level of the factors favourable to hyperplasia,
J94 the so-called ischemia reperfusion index expresses the level of tissular congestion relative to cellular metabolic activity,
J95 the so-called thrombogenic index expresses the risk of atherogenic transformation in the arterial walls; by extension, it allows one to evaluate the risk of thrombosis in the lumen,
J96 the so-called thrombotic index expresses the risk of thrombus development; by extension, it allows one to evaluate the level of blood coagulability,
J97 the so-called adjusted genital ratio index expresses the basal level of tissular androgenic activity relative to estrogenic activity, outside any acute adaptation phenomena,
J98 the so-called musculotropic index expresses the relative endocrinometabolic impact on muscle, in relation to the normal distribution of gonadic effects on muscle and bone,
J99 the so-called adjusted estrogenic index expresses the endocrinometabolic activity of the estrogens on the tissues; by extension, it bears witness to their potential level of specific activity within the structure of the organism,
J100 the so-called genital androgeny index expresses the endocrinometabolic activity of the gonadic androgens on the tissues; by extension, it bears witness to their potential level of specific activity within the structure of the organism,
J101 the so-called comparative genital androgeny index expresses the metabolic level of activity of the androgens' specific tissular targets; by extension, it bears witness to the functional level of activity of the androgens within the structure of the organism,
J102 the so-called “starter” index expresses the degree of hepatotropic stimulation by the splanchnic system, relative to splenotropic stimulation, in response to any kind of aggression, direct or indirect, on the tissues; by extension, it bears witness to the functional level of glucagon relative to that of adrenalin during the activation of the general adaptation syndrome, and consequently to the respective response threshold of both hormones to endogenous as well as exogenous stimulation; by extension, it bears witness to the level of priority of the energometabolic adaptative response relative to the neuroendocrine response,
J103 the so-called adjusted index of thyroid reactivating activity expresses the part played by endogenous factors in the stimulation of the thyrotropic axis by the locus coeruleus; by extension, it bears witness to the degree of endogenous maladaptation of the organism,
J104 the so-called pro-inflammatory index expresses the dysmetabolic potential of endogenous thyrotropic maladaptation and of its correction by cortisol; by extension, it bears witness to the degree of inflammatory stimulation of the structure in its unit of adaptability,
J105 the so-called index of inflammation expresses the actual level of inflammatory activity of the organism, of endogenous origin, in its present functional state; by extension, it bears witness to the threshold and to the gradient of the inflammatory reactivity of the individual,
J106 the so-called comparative index of inflammation expresses the degree of inflammation that is fundamental and structural relative to inflammation that expresses a functional and/or adaptative adjustment,
J107 the interleukin 1 index expresses the specific level of activity of interleukin 1, in its stimulation of growth as well as in its lymphocytic specificity,
J108 the dehydroepiandrosterone (DHEA) index expresses the level of DHEA endocrinometabolic activity; by extension, it permits the evaluation of its level of secretion and the degree of gonadotropic transformation,
J109 the serotonin index expresses the level of autacoid and metabolic activity of peripheral serotonin; by extension, it permits the evaluation of the level of neurometabolic activity of central serotonin,
J110 the so-called adjusted demyelinization index expresses the relative level of adaptability of the energometabolic response of insulin in its chronological relationship with the endocrine response of growth hormone,
J111 the so-called expansiveness index #1 expresses the potential level of cellular growth in its function of structural metabolic adaptation,
J112 the so-called expansiveness index #2 expresses the degree of risk of anarchic cellular development related to the adaptative modalities of the metabolism insofar as these have consequences with structural implications,
J113 the so-called global expansiveness index expresses the actual share of retained cellular expansion relative to the degree of destruction of pathological cells,
J114 the so-called ACTH index expresses the level of organometabolic activity of corticotropin,
J115 the so-called PTH index expresses the level of endocrinometabolic activity of parathormone,
J116 the so-called index of gonadotropic output expresses the endocrine activity of the gonadic estrogens relative to the level of stimulation by the pituitary; by extension, it helps one to evaluate the threshold of estrogenic response to hypophyseal stimulation; by extension, it helps one to evaluate the part that functional gonadic deficiency plays in the reactional activity of the hypophyseal gonadotrophins,
J117 the so-called index of pelvic congestion expresses the level of physiological pelvic congestion relative to pathological congestion,
J118 the so-called index of splanchnic congestion expresses the relative level of active congestion of the splanchnic reservoir; by extension, it situates the relative level of adapted congestion and of its reactive reduction,
J119 the so-called growth score index expresses the balanced resultant of the growth and anti-growth factors; by extension, it evaluates actual growth, organic as well as functional; by extension, it evaluates the potential for development of healthy or sick organs, as a result of factors structural, functional or adaptative (structural or functional adaptation),
J120 the so-called GH growth score index expresses the level that results from the endocrinometabolic activity of growth hormone; by extension, it evaluates the role played by the somatotropic axis in the general adaptation syndrome; by extension, it evaluates the role played by the somatotropic axis in the summoning and distribution of structural as well as of functional energy; by extension, it evaluates the degree to which growth hormone is responsible for the distribution of metabolic energy, whether in the sense of architecture, evolution or maintenance, or whether it be functional, chronological or adaptative,
J121 the so-called TRH/TSH ratio index expresses the level of TRH activity on the tissues—structural as well as adaptative—relative to TSH activity; by extension, it evaluates the level of congestion, compared to that of hyperplasia, in the process of anabolic adaptation; by extension, it evaluates the share of nutrition relative to metabolic output; by extension, it evaluates the share of neuroendocrine adaptation relative to organometabolic adaptation; by extension, it evaluates the share of potential elaboration relative to structural achievement; by extension, it evaluates the share of the imaginary relative to material realization,
J122 the so-called index of thyroid efficiency expresses the activity of the thyroid on the tissues as part of its structural activity,
J123 the so-called index of relative thyroid efficiency expresses the tissular metabolic activity of the thyroid relative to its actual metabolic activity,
J124 the so-called index of oxidation expresses the part played exclusively by the oxidation phase in the phenomenon of oxidation-reduction; by extension, it helps one to evaluate the level of cellular respiration,
J125 the so-called index of reduction expresses the part played exclusively by the reduction phase in the phenomenon of oxidation-reduction; by extension, it helps one to evaluate the level of the organism's antioxidant activity,
J126 the so-called pro-amyloid index expresses the level of intracellular hypometabolism; by extension, it evaluates the degree of cellular respiratory insufficiency; by extension, it evaluates the degree of cellular nutritional insufficiency,
J127 the so-called index of amyloid risk expresses the risk of amyloid degeneration of the organism; by extension, it evaluates the risk of intracerebral degenerative disease, and particularly of Parkinson's and Alzheimer's disease,
J128 the so-called index of thyroid output #2 expresses the tissular activity of the thyroid relative to the level of specific stimulation by the hypophyseal trophic hormones; by extension, it helps one to evaluate the thyroid's relative threshold of participation in cellular distribution,
J129 the so-called comparative index of thyroid output expresses the tissular activity of the thyroid relative to total metabolic activity; by extension, it helps one to evaluate the relative threshold of stimulation of the orientation activity of cellular distribution by the thyrotropic axis,
J130 the so-called index of estrogenic fraction #3 expresses the relative part played by the estrogens in endocrine tissular regulation within the gonadic apparatus,
J131 the so-called index of estrogenic fraction #4 expresses the extra adaptative endocrinometabolic cost incurred in the tissular activity of the estrogens,
J132 the so-called index of estrogenic fraction #5 expresses the relative share of the estrogens devoted to organotissular growth,
J133 the so-called general index of estrogenic fraction expresses the global share, structural as well as adaptative, of the estrogens devoted to organotissular growth,
J134 the so-called index of estrogenic fraction #6 expresses the endocrinometabolic activity of estrogens devoted to a structural structurofunctional mode of adaptation; by extension, it evaluates the level of gonadothyroid solidarity within the various adaptability units, and helps one to evaluate to what degree estrogenic demand is responsible for thyroid dysfunction,
J135 the so-called index of estrogenic fraction #7 expresses the extra adaptative endocrinometabolic cost of the activity of estrogens devoted to a structural structurofunctional mode of adaptation; by extension, it evaluates the endocrine cost of gonadothyroid solidarity within the various adaptability units, and it thus the reinjected cost of estrogenic demand in thyroid dysfunction,
J136 the so-called index of estrogenic fraction #8 expresses estrogenic endocrine activity within the gonadotropic adaptability unit; by extension, it evaluates the level of tissular anabolic/catabolic equilibrium and of tissular bone-remodeling equilibrium,
J137 the so-called general quantitative estrogenic index expresses the final quantitative activity of the estrogens at the cellular and tissular level; by extension, it evaluates the proteic yield, and hence the level of nuclear activity relative to cellular and then to tissular activity,
J138 the so-called index of specific estrogenic fraction expresses the musculotropic activity of the estrogens; by extension, it evaluates the level of estrogenic endocrine activity as part of the gonadic activity,
J139 the so-called comparative estrogenic index #1 expresses the share of estrogenic activity devoted to organotissular growth relative to total endocrinometabolic activity,
J140 the so-called comparative estrogenic index #2 expresses the share of estrogenic activity devoted to organotissular growth relative to total endocrinometabolic tissular activity,
J141 the so-called global comparative estrogenic index expresses the share of estrogenic activity devoted to organotissular growth—whether it be an adaptative growth of restoration (as well as of function) or a basal structural growth or a growth of adaptability,
J142 the so-called index of somatotropic estrogenic output expresses the organotissular growth activity of the peripheral estrogens relative to the global activity of pituitary FSH,
J143 the so-called index of quantitative organotissular estrogenic output expresses the organotissular activity of the peripheral estrogens relative to the global activity of pituitary FSH,
J144 the so-called FSH index #1 expresses the organometabolic activity of pituitary follicle-stimulating hormone,
J145 the so-called LH index#1 expresses the endocrinometabolic activity of pituitary luteinizing hormone,
J146 the so-called FSH index #2 expresses the endocrinometabolic activity of pituitary follicle-stimulating hormone,
J147 the so-called LH index #2 expresses the organometabolic activity of pituitary luteinizing hormone,
J148 the so-called index of progesterone output expresses the gonadic endocrine activity of progesterone relative to that of estrogen,
J149 the so-called ketonic index expresses the level of prevalence of glucagon over the adrenal-insulinic reaction; by extension, it allows one to evaluate the role played, in the management of glucide-derived energy, by the ketonic route relative to the insulinic and to the cortico-adrenergic route,
J150 the so-called index of total subliminal TRH expresses the threshold of reactivity of TRH in its thyrotropic endocrine activity (hypophyseal and thyroid), as well as in its thyrosomatotropic metabolic activity,
J151 the so-called index of active carcinogenesis expresses the actual carcinogenetic level of the organism, retained and related to the degree of destruction of pathological cells (through apoptosis or necrosis),
J152 the so-called comparative index of active carcinogenesis expresses the actual functional level of the carcinogenesis within the organism, retained and related to the degree of destruction of pathological cells (through apoptosis or necrosis),
J153 the so-called gonadothyrotropic index expresses the part played by the gonads in the appeal to and the response by the thyroid, both metabolic and endocrine, in the functional adaptation of structure,
J154 the so-called index of global tissular estrogenic fraction expresses the relative share of estrogenic activity devoted entirely to the tissues.

The software product according to the invention can be used with a computer, either an analog one or a digital one. In practice, it is loaded either directly in the computer memory or indirectly by means of a software interface or of an intermediary module, the aforesaid memory being (preferably) internal or auxiliary.

Whatever the operating mode, the configuration, the structure or the operating system of the computer, it is advisable to store up the software product on a computer readable medium. It comprises computer readable software means which are needed to execute steps (3°) and (4°) of the method according to the invention.

In practice, said software product comprises:
(A) a form field for entering (i) the patient's name or code, (ii) his age, and (iii) his sex, on the one hand, and any known treatment followed by said patient formerly or at the present time, on the other hand;
(B) a form field for entering (iv) one or several hematic parameters measured in vitro in the patient's blood and (v) the date of the measures;
(C) a field including all the indexes from J1 to J157, (vii) their calculation mode and (viii) their median values determined from human beings who are recognized as healthy subjects;
(D) a command for (ix) the calculation of one index J, of several indexes J or the totality of indexes from the hematic parameters of said field (B) obtained from the blood of the patient to be tested, and (x) for the comparison of the obtained value for at least one of said indexes J with its median value of field (C); and,
(E) means for classifying, visualizing, editing and/or printing the obtained result by implementing command (D) starting from fields (A), (B), and (C).

Field (A) is designed for the input of the data that are essential and necessary to identify the patient (his name or his code number, his age), and of the data allowing the improvement of the comparison analysis according to step (4°) of the method of the invention (followed treatment).

It is recommended to note the blood sampling date and (if it is different) the determination date of the parameter(s) of step (2°).

Each median value of an index J comprises an interval of normal values previously reckoned among adults who are healthy subjects.

Moreover, it is advisable to include in the software product according to the invention an instruction for the reckoning and flagging up of any abnormality consisting of a value of an index J which stands away from the median value or from its interval.

Other advantages or characteristics of the invention will be better understood when reading the following implemented examples. Of course, these examples are by no means limitative, but they are given as illustration purpose.

In these examples, when the determination (“typing”) of one or several parameters of step (2°) is stated at times T₀, T₁, T₂, T₃, etc., it is proper to recall that the time intervals T₁-T₀, T₂-T₁, T₃-T₂, etc. between two determinations, depend on the patient and on the evolution of his pathology; in other words these time intervals are variable, on the average they last 2, 3, 4, and even 6 months.

### EXAMPLE 1

### Menstruation Score

The menstruation score comprises, among others:

A very noticeable increase (see Table I) in:

the necrosis index (J45)
the oxidation-reduction index (J67)
the index of oxidation (J124)
the index of free radicals (J82)
the thrombotic index (J96), and

the level of catabolism (J31):

TABLE I
MENSTRUATION SCORE
INCREASE OF J45, J67, J124, J82, J96 et J31

| | | | Date | | | | | |
|---|---|---|---|---|---|---|---|---|
| Control | | | 99/10/02 | 00/09/09 | 99/09/07 | 00/6/19 | 99/09/21 | 97/09/09 |
| (normal | | | Day of the cycle | | | | | |
| interval) | | | 3 d/30 d | 1 d/29 d | 3 d/28 d | 1 d/28 d | 2 d | 3 d/32 d |
| Mini | Maxi | Name | D. Murielle | D. Murielle | G. Véronique | G. Véronique | M. Danila | D. Cécile |
| 2.5 | 6 | J45 | 22.97 | 24.21 | 109.82 | 100.63 | 9.6 | 77.27 |
| | | | Date | | | | | |
| | | | 99/10/02 | 00/09/09 | 99/09/07 | 00/6/19 | 99/09/21 | 97/09/09 |
| | | | Day of the cycle | | | | | |
| Control | | | 3 d/30 d | 1 d/29 d | 3d /28 d | 1 d/28 d | 2 d | 3 d/32 d |
| Mini | Maxi | Name | D. Murielle | D. Murielle | G. Véronique | G. Véronique | M. Danila | D. Cécile |
| 0.7 | 2 | J67 | 20257 | 7720 | 29391 | 72334 | 1215 | 4707 |
| 1.44 | 81 | J124 | 11309 | 5917.86 | 1965 | 2784 | 1384.76 | 817.01 |
| 0.25 | 0.6 | J82 | 842.49 | 382.92 | 2205 | 7336 | 94.39 | 408.16 |
| 4 | 8 | J96 | 24.53 | 25.25 | 10.26 | 16.60 | 3.50 | 1.05 |
| 1.3 | 1.6 | J31 | 0.75 | 0.77 | 2.64 | 1.93 | 0.73 | 1.35 |

And a very noticeable decrease (see Table II) for:
the index of reduction (J125)
the fibrosis index (J8)
the amylosis index (J72)

the level of anabolism (J32), and the prolactin index (J39).

TABLE II
MENSTRUATION SCORE, INDEXES DECREASE
J125, J8, J72, J32 and J39

| | | | Date | | | | | |
|---|---|---|---|---|---|---|---|---|
| | | | 99/10/02 | 00/09/09 | 99/09/07 | 00/6/19 | 99/09/21 | 97/09/09 |
| | | | Day of the cycle | | | | | |
| Control | | | 3 d/30 d | 1 d/29 d | 3 d/28 d | 1 d/28 d | 2 d | 3 d/32 d |
| Mini | Maxi | Name | D. Murielle | D. Murielle | G. Véronique | G. Véronique | M. Danila | D. Cécile |
| 0.72 | 116 | J125 | 0.56 | 0.77 | 0.07 | 0.04 | 1.14 | 0.17 |
| 6 | 8 | J8 | 0.28 | 0.43 | 1.52 | 0.48 | 0.68 | 3.01 |
| 10 | 17 | J72 | 0.56 | 0.66 | 1.15 | 0.26 | 0.35 | 0.11 |
| 0.65 | 0.8 | J32 | 0.22 | 0.23 | 1.48 | 0.77 | 0.21 | 0.48 |
| 0.8 | 1.2 | J39 | 0.20 | 0.20 | 0.24 | 0.10 | 0.13 | 0.05 |

### EXAMPLE 2

### Ovulation Score

The ovulation score comprises among others:

A very noticeable increase (see Table III) for:
the prolactin index (J39)
the index of circulating cortisol (J26)
all the tissular indexes of estrogens (J19, J134, J135)
the index of reduction (J125)
the fibrosis index (J8)
the amylosis index (J72), and
the level of anabolism (J32);
   for the same sample of observations, this increase is shown in Table III by the ratio (O/M) of the basic values during the cycle (O) relative to the values during or around menstruation (M); and

A very noticeable decrease (see Table IV) for:

the indexes of metabolic activity of estrogens (J20 et J138).

TABLE III
OVULATION SCORE, INDEXES INCREASE

| | Ratio | | | |
|---|---|---|---|---|
| | O/M | O/M | O/M | O/M |
| | Name | | | |
| Index | D. Murielle | G. Véronique | M. Danila | D. Cécile |
| J39 | 5.03 | 1.62 | 1.70 | 10.75 |
| J26 | 1.26 | 2.18 | 2.29 | 0.52 |
| J134 | 2.67 | 2.40 | 1.08 | 1.06 |
| J135 | 1.29 | 1.22 | 1.03 | 1.54 |
| J19 | 1.23 | 1.07 | 1.02 | 1.47 |
| J125 | 57.00 | 1000.85 | 24.31 | 457.51 |
| J8 | 21.93 | 4.93 | 3.87 | 3.17 |
| J72 | 8.82 | 13.06 | 1.58 | 30.73 |
| J32 | 1.41 | 0.06 | 0.69 | 0.36 |

TABLE IV
OVULATION SCORE, INDEXES DECREASE

| | Ratio | | | |
|---|---|---|---|---|
| | O/M | O/M | O/M | O/M |
| | Name | | | |
| Index | D. Murielle | G. Véronique | M. Danila | D. Cécile |
| J138 | 0.32 | 2.48 | 1.47 | 2.16 |
| J20 | 0.42 | 0.59 | 0.85 | 1.22 |

### EXAMPLE 3

### Seasonal Syndrome

The pre-seasonal autumn syndrome is analyzed below. It bears witness to the metabolic summoning (i.e. implementing or mobilization of the biological potential involved) essential for winter structuro-functional equilibrium. It is an adaptability period which implies putting in place the general adaptative crossroads, immediate mobilization of the thyrosomatotropic axis and preparation of the functional increase of the gonadotropic axis. It is expressed by the pre-autumn score. It comprises, among others:

A clear increase of:
on the adaptative level (see Table V):
   the “starter” index (J102)
   the index of potential histamine (J88)
   the prolactine index (J39)
   the index of active histamine (J87)
   the index of circulating cortisol (J26)

the index of corticoadrenal adaptation/permissiveness (J68);

TABLE V

Control

| Mini | Maxi | Indexes | Obtained data |
|---|---|---|---|
| 0.85 | 1.15 | J102 | 1.30 |
| 6 | 12 | J88 | 630.70 |
| 0.8 | 1.2 | J39 | 1.40 |
| 20 | 60 | J87 | 168.87 |
| 3 | 7 | J26 | 7.51 |
| 1 | 3 | J68 | 5.09 |

on the strictly thyrotropic thyrosomatotropic level (see Table VI):
   the index of thyroid reactivating activity (J16)
   the adjusted index of thyroid reactivating activity (J103)
   the index of thyroid output #1 (J81)
   the TRH/TSH ratio index (J121)
   the adenosis index (J93);

the index of thyroid involvement (J9);

TABLE VI

Control

| Mini | Maxi | Indexes | Collected data |
|---|---|---|---|
| 0.05 | 0.25 | J16 | 0.28 |
| 0.1 | 0.5 | J103 | 0.62 |
| 2 | 3 | J81 | 3.10 |
| 0.33 | 1.70 | J121 | 4.75 |
| 10 | 30 | J93 | 40.98 |
| 4 | 8 | J9 | 27.78 |

on the specific thyrosomatotropic level (see Table VII):
   the amylosis index (J72)
   the index of amylosis risk (J73)
   the pro-amyloid index (J126)
   the index of amyloid risk (J127)
   the index of insulin resistance (J74)
   the fibrosis index (J8)
   the insulin index (J54)

the demyelinization index (J55):

TABLE VII

Control

| Mini | Maxi | Indexes | Observed data |
|---|---|---|---|
| 10 | 17 | J72 | 25.22 |
| 5 | 8 | J73 | 30541.88 |
| 0.54 | 145.50 | J126 | 523.80 |
| 0.07 | 5.56 | J127 | 92.10 |
| 0.75 | 1.25 | J74 | 2.27 |
| 6 | 8 | J8 | 17.87 |
| 1.5 | 5 | J54 | 8.83 |
| 5 | 15 | J55 | 23.11 |

on the gonadotropic level (see Table VIII):
   the folliculin index (J53)
   the index of somatotropic estrogenic output (J142)
   the level of activity of genital estrogens (J50)
   the androgenic index (J27)
   the level of activity of total androgens (J46)
   the rate of genital androgens (J48)
   the adjusted estrogenic index (J99)
   the genital androgeny index (J100)

the comparative genital androgeny index (J101);

TABLE VIII

Control

| Mini | Maxi | Indexes | Observed data |
|---|---|---|---|
| 0.75 | 1.25 | J53 | 1.80 |
| 2.14 | 206.25 | J142 | 7369.90 |
| 1.2 | 2 | J27 | 2.82 |
| 0.2 | 0.25 | J46 | 0.25 |
| 0.12 | 0.17 | J48 | 0.27 |
| 0.12 | 0.16 | J50 | 0.18 |
| 0.2 | 0.5 | J99 | −0.65 |
| 0.05 | 0.09 | J100 | −1.86 |
| 0.1 | 0.3 | J101 | 1.90 |

on the metabolic level (see Table IX):
   the index of active cellular permeability (J61)
   the index of adjusted active cellular permeability (J62)
   the index of passive cellular permeability (J63)
   the index of active intracellular osmolar gradient (J64)
   the index of adjusted active intracellular osmolar gradient (J65)
   the insulin index (J54)
   the adjusted growth index (J36)
   the oxidation-reduction index (J67)
   the index of oxidation (J124)
   the index of free radicals (J82)
   the adjusted index of free radicals (J83)
   the comparative index of free radicals (J84)
   the index of free radical nocivity (J85)
   the level of anabolism (J32)
   the index of pelvic congestion (J117)
   the index of splanchnic congestion (J118)
   the serotonin index (J109)
   the index of insulin resistance (J74)
   the level of metabolic activity efficiency (J33)

the level of catabolism (J31);

TABLE IX

Control

| Mini | Maxi | Indexes | Collected data |
|---|---|---|---|
| 6 | 9 | J61 | 15.38 |
| 0.8 | 1 | J62 | 1.72 |
| 4 | 9 | J63 | 57.33 |
| 8 | 12 | J64 | 81.12 |
| 1 | 1.5 | J65 | 5.16 |
| 1.5 | 5 | J54 | 8.83 |
| 0.06 | 0.1 | J36 | 0.15 |
| 0.7 | 2 | J67 | 154827.17 |
| 1.44 | 81.48 | J124 | 1905.92 |
| 0.25 | 0.6 | J82 | 8913.29 |
| 1.8 | 3.5 | J83 | 8896.86 |
| 2 | 4 | J84 | 8894.56 |
| 2 | 6 | J85 | 45789.68 |
| 0.65 | 0.8 | J32 | 1.17 |
| 0.01 | 0.19 | J117 | 1.50 |
| 0.01 | 0.16 | J118 | 1.62 |
| 1.5 | 7.5 | J109 | 14.50 |
| 0.75 | 1.25 | J74 | 2.27 |
| 80 | 1.40 | J33 | 334 |
| 1.3 | 1.6 | J31 | 1.98 |

on the pathogenesis level (see Table X):
   the necrosis index (J45)
   the level of membrane expansion (J40)
   the level of membrane fracture (J44)
   the index of nucleocytoplasmic pathogenicity (J57)
   the index of carcinogenesis (J59)
   the index of comparative carcinogenesis (J60)
   the expansiveness index #1 (J111)
   the expansiveness index #2 (J112)
   the level of structural expansion (J41)
   the index of cancer potential (J92)
   the index of cellular fracture (J58)
   the index of pelvic congestion (J117)
   the index of splanchnic congestion (J118)
   the pro-inflammatory index (J104)
   the index of inflammation (J105)
   the comparative index of inflammation (J106)

the thrombotic index (J96);

TABLE X

Control

| Mini | Maxi | Indexes | Observed data |
|---|---|---|---|
| 2.5 | 6 | J45 | 27.75 |
| 0.08 | 0.16 | J40 | 0.25 |
| 1.5 | 1.9 | J44 | 4.54 |
| 0.8 | 1.5 | J57 | 1.55 |
| 1 | 3 | J59 | 9.76 |
| 1 | 1.5 | J60 | 2.40 |
| 0.06 | 2 | J111 | 2.11 |
| 1 | 4 | J112 | 4.72 |
| 0.04 | 0.08 | J41 | 0.09 |
| | 10 | J92 | 2154166 |
| 0.5 | 1.5 | J58 | 1.69 |
| 0.01 | 0.19 | J117 | 1.50 |
| 0.01 | 0.16 | J118 | 1.62 |
| 0.1 | 0.4 | J104 | 0.42 |
| 0.3 | 2.5 | J105 | 11.24 |
| 0.2 | 2.5 | J106 | 10.39 |
| 4 | 8 | J96 | 26.92 |

A clear decrease (see Table XI) in:
the turn over index (J7)
the catabolism/anabolism ratio index (J25), and a relative decrease in:

the musculotropic index (J98);

TABLE XI

Control

| Mini | Maxi | Indexes | Collected data |
|---|---|---|---|
| 40 | 60 | J7 | 39.3 |
| 1.8 | 3 | J25 | 1.72 |
| 0.53 | 4.72 | J98 | 4.25 |

In summer, the musculotropic index rises up to 12.87, the ratio of pre-automn/summer values is therefore much lower than 1; it is equal to 0.33.

### EXAMPLE 4

### Seasonal Score

The pre-seasonal spring syndrome is analyzed below. It bears witness to the metabolic summoning essential for the summer structuro-functional equilibrium. It is an adaptability period which implies putting in place the general adaptation crossroads, immediate mobilization of the thyrosomatotropic axis and preparation of the functional decrease of the gonadotropic axis. It is expressed by the pre-spring score. It comprises, among others:

A clear increase in:
at the adaptative level (see Table XII):
   the “starter” index (J102)
   the index of potential histamine (J88)
   the prolactine index (J39)
   the index of active histamine (J87)
   the ACTH index (J114)
   the DHEA index (J108)
   the adaptation index (J3)
   the index of circulating cortisol (J26)

the adrenal cortex index (J28);

TABLE XII

Control

| Mini | Maxi | Indexes | Observed data |
|---|---|---|---|
| 0.85 | 1.15 | J102 | 1.32 |
| 6 | 12 | J88 | −73.91 |
| 0.8 | 1.2 | J39 | 1.40 |
| 20 | 60 | J87 | 295.00 |
| 0.71 | 3.00 | J114 | 107.07 |
| 5 | 9 | J108 | 132.24 |
| 0.25 | 0.5 | J3 | 0.58 |
| 3 | 7 | J26 | 7.32 |
| 2.7 | 3.3 | J28 | 3.59 |

at the strictly thyrotropic thyrosomatotropic level (see Table XIII):
   the index of thyroid involvement (J9);
   the index of thyroid reactivating activity (J16)
   the adjusted index of thyroid reactivating activity (J103)
   the TRH/TSH ratio index (J121)

the adenosis index (J93);

TABLE XIII

Control

| Mini | Maxi | Indexes | Collected data |
|---|---|---|---|
| 0.05 | 0.25 | J16 | 0.27 |
| 0.1 | 0.5 | J103 | 0.65 |
| 0.33 | 1.70 | J121 | 5.69 |
| 4 | 8 | J9 | 39.00 |
| 10 | 30 | J93 | 63.35 |

at the specific thyrosomatotropic level (see Table XIV):
   the amylosis index (J72)
   the index of insulin resistance (J74)
   the level of membrane expansion (J40)
   the insulin index (J54)
   the fibrosis index (J8)
   the adjusted growth index (J36)
   the level of structural expansion (J41)
   the prolactin index (J39)

the interleukin 1 index (J107);

TABLE XIV

Control

| Mini | Maxi | Index | Observed data |
|---|---|---|---|
| 10 | 17 | J72 | 29.77 |
| 0.75 | 1.25 | J74 | 1.41 |
| 0.08 | 0.16 | J40 | 0.17 |
| 1.5 | 5 | J54 | 4.92 |
| 6 | 8 | J8 | 13.02 |
| 0.06 | 0.1 | J36 | 0.13 |
| 0.04 | 0.08 | J41 | 0.06 |
| 0.8 | 1.2 | J39 | 1.40 |
| 0.1 | 0.16 | J107 | 0.20 |

at the gonadotropic level (see Table XV):
   the folliculin index (J53)
   the index of somatotropic estrogenic output (J142)
   the FSH index #1 (J144)
   the LH index #1 (J145)
   the genital androgeny index (J100)
   the comparative genital androgeny index (J101)
   the androgenic index (J27)
   the level of activity of total androgens (J46)
   the rate of genital androgens (J48)

the level of activity of genital estrogens (J50)

TABLEAU XV

| Control | | |
|---|---|---|
| Maxi | Indexes | Observed data |
| 1.25 | J53 | 1.33 |
| 206.25 | J142 | 633.62 |
| 12.00 | J144 | 76.38 |
| 6.00 | J145 | 61.44 |
| 0.09 | J100 | 0.35 |
| 0.3 | J101 | 0.88 |
| 2 | J27 | 2.76 |
| 0.25 | J46 | 0.30 |
| 0.17 | J48 | 0.21 |
| 0.16 | J50 | 0.19 |

at the metabolic level (see Table XVI):
   the index of active cellular permeability (J61)
   the index of adjusted active cellular permeability (J62)
   the index of passive cellular permeability (J63)
   the index of active intracellular osmolar gradient (J64)
   the index of passive intracellular osmolar gradient (J66)
   the insulin index (J54)
   the adjusted growth index (J36)
   the level of anabolism (J32)
   the oxidation-reduction index (J67)
   the index of oxidation (J124)
   the index of free radicals (J82)
   the adjusted index of free radicals (J83)
   the comparative index of free radicals (J84)
   the index of free radical nocivity (J85)
   the level of catabolism (J31);
the serotonin index (J109)
   the index of insulin resistance (J74)
   the prolactin index (J39)
   the level of metabolic activity efficiency (J33)

the index of reduction (J125);

TABLE XVI

Control

| Mini | Maxi | Indexes | Observed data |
|---|---|---|---|
| 6 | 9 | J61 | 16.40 |
| 0.8 | 1 | J62 | 4.31 |
| 4 | 9 | J63 | 20.37 |
| 8 | 12 | J64 | 10.48 |
| 1 | 3.3 | J66 | 3.92 |
| 1.5 | 5 | J54 | 4.92 |
| 0.06 | 0.1 | J36 | 0.13 |
| 0.65 | 0.8 | J32 | 0.89 |
| 0.7 | 2 | J67 | 2515.12 |
| 1.44 | 81.5 | J124 | 233.09 |
| 0.25 | 0.6 | J82 | 468.02 |
| 1.8 | 3.5 | J83 | 464.86 |
| 2 | 4 | J84 | 466.58 |
| 1.70 | 6.00 | J85 | 100039.73 |
| 1.3 | 1.6 | J31 | 1.68 |
| 1.5 | 7.5 | J109 | 16.83 |
| 0.75 | 1.25 | J74 | 1.41 |
| 0.8 | 1.2 | J39 | 1.40 |
| 80 | 140 | J33 | 114.25 |
| 0.7 | 116 | J125 | 271.91 |

at the pathogenesis level (see Table XVII):
   the index of free radical nocivity (J85)
   the necrosis index (J45)
   the oxidation-reduction index (J67)
   the index of cellular fracture (J58)
   the level of membrane expansion (J40)
   the level of membrane fracture (J44)
   the fibrosis index (J8)
   the index of cancer potential (J92)
   the index of carcinogenesis (J59)
   the level of structural expansion (J41)
   the index of comparative carcinogenesis (J60)
   the index of DNA fracture (J56)
   the expansiveness index #1 (J111)
   the expansiveness index #2 (J112)
   the index of nucleocytoplasmic pathogenicity (J57)
   the index of carcinogenic expansion (J91)
   the adenosis index (J93)
   the amylosis index (J72)
   the index of amylosis risk (J73)
   the index of potential histamine (J88)
   the pro-inflammatory index (J104)
   the index of inflammation (J105)
   the comparative index of inflammation (J106)
   the index of splanchnic congestion (J118)
   the thrombotic index (J96)
   the pro-amyloid index (J126)
   the index of amyloid risk (J127);

And a clear decrease in:

the βMSH/αMSH index (J70) (see Table XVIII)

TABLE XVII

Control

| Mini | Maxi | Indexes | Observed data |
|---|---|---|---|
| 0.08 | 0.16 | J40 | 0.32 |
| 0.04 | 0.08 | J41 | 0.18 |
| 2.5 | 6 | J45 | 9.69 |
| 6 | 8 | J8 | 13.02 |
| 0.5 | 1.5 | J56 | 1.62 |
| 0.8 | 1.5 | J57 | 3.43 |
| 1.5 | 1.9 | J44 | 3.96 |
| 0.5 | 1.5 | J58 | 2.12 |
| 1 | 3 | J59 | 43.36 |
| 1 | 1.5 | J60 | 2.49 |
| 0.7 | 2 | J67 | 2515.12 |
| 10 | 17 | J72 | 29.77 |
| 5 | 8 | J73 | 45178.57 |
| 1.70 | 6.00 | J85 | 100039.73 |
| 6 | 12 | J88 | −73.91 |
| 0.3 | 1 | J91 | 2.70 |
| 6 | 10 | J92 | 6436.12 |
| 10 | 30 | J93 | 63.35 |
| 5 | 8 | J96 | 26.92 |
| 0.1 | 0.4 | J104 | 0.59 |
| 0.3 | 2.5 | J105 | 5.83 |
| 0.2 | 2.5 | J106 | 5.38 |
| 0.06 | 2 | J111 | 2.95 |
| 1 | 4 | J112 | 8.32 |
| 0.01 | 0.19 | J117 | 2.30 |
| 0.01 | 0.16 | J118 | 2.06 |
| 0.5 | 146 | J126 | 423.03 |
| 0.1 | 6 | J127 | 14.45 |

TABLE XVIII

Control

| Mini | Maxi | Indexes | Collected data |
|---|---|---|---|
| 6 | 8 | J70 | 5.04 |

### EXAMPLE 5

### Characteristic Variations of Specific Indexes

One notices, among others, a characteristic increase in the fibrosis index (J8) during evolutionary phases, toward cirrhosis, of hepatic degenerative affections, particularly during viral hepatitis and evolutionary phases of pulmonary fibrosis during spurts of chronic bronchitis.

Table XIX below shows the regressive evolution of the fibrosis index (J8), under the influence of endobiogenic treatment, with a patient suffering from hepatitis C, between time T₀ and time T₃.

TABLE XIX

| | |
|---|---|
| Control | Observed data |

| Mini | Maxi | Indice | T₀ | T₁ | T₂ | T₃ |
|---|---|---|---|---|---|---|
| 6 | 8 | J8 | 14.5 | 14.3 | 6.9 | 3.8 |

One notices an important increase in the apoptosis index (J42) during evolutionary spurts and viral replication of the main viral diseases such as AIDS, viral hepatitis, influenza, and during characteristic fits of radical overactivity.

Table XX below shows the regressive evolution of the apoptosis indexes (J42 and J43) under the influence of a triple therapy treatment with a patient suffering from AIDS, at time T₀, T₁, T₂, T₃, T₄ and T₅.

TABLE XX

| Control | | | Observed data | | | | | |
|---|---|---|---|---|---|---|---|---|
| Mini | Maxi | Indexes | T₀ | T₁ | T₂ | T₃ | T₄ | T₅ |
| 0.3 | 0.7 | J42 | 6638 | 5827 | 623049 | 364647 | 525 | 91 |
| 5 | 8 | J43 | 3723 | 2194 | 20229 | 3897 | 412 | 187 |

One also notices an increase in the index of insulin resistance (J74) during setting in phases and evolutionary phases of non-insulin-dependent diabetes.

In Table XXI below, one has put down the regressive evolution of the index of insulin resistance (J74) under the influence of endobiogenic treatment and diet with obese and pre-diabetic patients, at time T₀, T₁, T₂ and T₃.

TABLE XXI

| | Control | | | Observed data | | | |
|---|---|---|---|---|---|---|---|
| Patient | Mini | Maxi | Index | T₀ | T₁ | T₂ | T₃ |
| 1 | 0.75 | 1.25 | J74 | 3.15 | 1.69 | 0.47 | 0.37 |
| 2 | 0.75 | 1.25 | J74 | 30.15 | 8.51 | 2.41 | |
| 3 | 0.75 | 1.25 | J74 | 12.89 | 9.80 | 1.05 | 0.95 |
| 4 | 0.75 | 1.25 | J74 | 34.7 | 21.88 | 0.30 | 0.19 |
| 5 | 0.75 | 1.25 | J74 | 3061 | 143 | | |
| 6 | 0.75 | 1.25 | J74 | 1.83 | 1.04 | | |
| 7 | 0.75 | 1.25 | J74 | 23.9 | 3.86 | 0.44 | 0.16 |
| 8 | 0.75 | 1.25 | J74 | 9.12 | 3.58 | 0.81 | |

One also notices an increase of the insulin index (J54) during setting in phases, as well as during chronic phases of obesity (see Table XXII).

TABLE XXII

| | | | | Observed data | | | |
|---|---|---|---|---|---|---|---|
| Patient | Control | | Index | T₀ | T₁ | T₂ | T₃ |
| 9 | 1.5 | 5 | J54 | 4.56 | 51.16 | | |
| 10 | 1.5 | 5 | J54 | 49.07 | | | |
| 11 | 1.5 | 5 | J54 | 3.09 | 7.88 | 9.22 | 90.81 |
| | | 5 | J54 | 4.25 | 9.96 | | |

One also notices an increase of the index of cancer potential (J92) and of the adenosis index (J93) during setting in and chronic phases of adenomyosis of the uterus or of the prostate, dysplasia, scrofula, and regression of mitotic neoplasia activity (see Table XXIII).

TABLE XXIII

| | Control | | | Observed data | | | | |
|---|---|---|---|---|---|---|---|---|
| Patient | Mini | Maxi | Indexes | T₀ | T₁ | T₂ | T₃ | T₄ |
| 13 | 10 | 30 | J93 | 3409 | 2143 | 617.87 | 1629 | 2117.60 |
| | 6 | 10 | J92 | 0.011 | 0.028 | 0.4315 | 0.172 | 0.05 |
| 14 | 10 | 30 | J93 | 407 | 223 | 1036 | 2118 | 407.42 |
| | 6 | 10 | J92 | 0.68 | 1.07 | 482 | 0.05 | 0.68 |
| 15 | 10 | 30 | J93 | 223 | 1036 | 2118 | 407 | 223.18 |
| | 6 | 10 | J92 | 1.07 | 482 | 0.05 | 0.68 | 1.07 |
| 16 | 10 | 30 | J93 | 54.53 | 510 | 3921.9 | 68715 | 2447.87 |
| | 6 | 10 | J92 | 0.401 | 0.009 | 0.0027 | | |
| 17 | 10 | 30 | J93 | 18.61 | 34.03 | 25.87 | 23.35 | |
| | 6 | 10 | J92 | 1.37 | 0.19 | 1.75 | 7.82 | |
| 18 | 10 | 30 | J93 | 433.5 | 581.4 | 372.13 | 34.71 | 312.21 |
| | 6 | 10 | J92 | 1.621 | 9.099 | 3.9416 | 4.795 | 0.34 |
| 19 | 10 | 30 | J93 | 162 | 197.7 | 215.23 | 192 | |
| | 6 | 10 | J92 | 47 | 6.781 | 19.049 | 0.82 | |
| 20 | 10 | 30 | J93 | 29.46 | 1155 | 316.99 | 58.67 | 35.61 |
| | 6 | 10 | J92 | 2.45 | 0.14 | 2.31 | 1.34 | 3.94 |
| 21 | 10 | 30 | J93 | 16.94 | 25.56 | | | |
| | 6 | 10 | J92 | 12.47 | 5.62 | | | |
| 22 | 10 | 30 | J93 | 24.82 | 2680 | | 47.96 | |
| | 6 | 10 | J92 | 3.92 | 0.87 | 0.48 | 3.37 | 0.64 |

One notices an increase of the circulating cortisol index (J26) and of the adrenal cortex index (J28) during all the acute phases of the summoning up of the adaptation syndrome, as well as during the prolonged phases whatever the nature of the infectious, meteorological, psychological or traumatic aggression (see Table XXIV)

TABLE XXIV

| | Control | | | Observed data | | | | |
|---|---|---|---|---|---|---|---|---|
| Patient | Mini | Maxi | Indexes | T₀ | T₁ | T₂ | T₃ | T₄ |
| 23 | 3 | 7 | J26 | 13.61 | 11.18 | | | |
| | 2.7 | 3.3 | J28 | 2.392 | 3.207 | | | |
| 24 | 3 | 7 | J26 | 36.88 | 11.2 | 7.89 | 37.07 | 23.35 |
| | 2.7 | 3.3 | J28 | 12.33 | 5.823 | 2.84 | 13.47 | 5.84 |
| 25 | 3 | 7 | J26 | 6.27 | 3.22 | 1.7115 | 10.94 | 39.47 |
| | 2.7 | 3.3 | J28 | 1.658 | 0.62 | 1.7768 | 0.883 | 1.98 |

On the other hand, one notices a characteristic decrease in the adenosis index and of the apoptosis index (J42) during evolutionary spurts of neoplasia affections. (see Table XXV).

TABLEAU XXV

| | Control | | | Observed data | | | | |
|---|---|---|---|---|---|---|---|---|
| Patient | Mini | Maxi | Index | T₀ | T₁ | T₂ | T₃ | T₄ |
| 26 | 0.3 | 0.7 | J42 | 0.61 | 0.44 | 0.34 | 0.23 | 0.12 |
| 27 | 0.3 | 0.7 | J42 | 0.54 | 0.34 | 0.25 | 0.16 | 0.09 |
| 28 | 0.3 | 0.7 | J42 | 0.43 | 0.40 | 0.37 | 0.07 | |
| 29 | 0.3 | 0.7 | J42 | 0.48 | 0.20 | 0.18 | 0.16 | 0.13 |
| 30 | 0.3 | 0.7 | J42 | 0.28 | 0.22 | 0.20 | | |
| 31 | 0.3 | 0.7 | J42 | 0.56 | 0.31 | 0.28 | 0.21 | 0.20 |

One also observes a noticeable decrease in the fibrosis index (J8) during post-cicatricial tissular regeneration phases after surgical interventions, on one hand, and of the insulin (J54) index during setting in as well as evolutionary phases of insulin dependent as well as non insulin dependent diabetes, on the other hand.

1. A method for evaluating the dynamic biological state of a patient, said method comprising, measuring several elements or substances present in blood and interpreting results of performed measures, comprising the following steps:
(1°) providing the blood previously taken from a patient;
(2°) determining in vitro, from said blood, hematic substances as metabolic and/or tissular parameters:
number of red blood cells (GR),
number of leukocytes (GB),
hemoglobin (HG),
number of neutrophils,
number of eosinophils,
number of lymphocytes,
number of monocytes,
number of platelets,
lactate dehydrogenase (LDH),
creatine phosphokinase (CPK),
thyroid-stimulating hormone (TSH),
alkaline phosphatases,
liver (H1 and H2), bone (01) and/or intestine (I1, I2 and I3) isoenzymes,
osteocalcin,
potassium and calcium, and optionally, at least one of the following substances:
carcinoembryonic antigen (CEA),
one or several CA15-3, CA125 and CA19-9 markers,
acid phosphatases, in particular prostatic acid phosphatase (PAP),
prostate specific antigen (PSA),
hourly sedimentation rate (ESR₁),
bihourly sedimentation rate (ESR₂),
thyroid hormones, in particular triiodothyronine (FT3) and thyroxine (FT4),
g-glutamyl transpeptidases,
transaminases,
chlorides and sodium, and
adrenocorticotropic hormone (ACTH);
(3°) measuring, from step (2°), at least one index selected from the group consisting of following indexes J1-J157:
J1 a so-called genital ratio index, which is a ratio red blood cells/leukocytes,
J2 a so-called genital-thyroid ratio index, which is a ratio neutrophils/lymphocytes,
J3 a so-called adaptation index, which is a ratio eosinophils/monocytes,
J3 being such that J3=eosinophils/monocytes=ACTH/FSH,
J4 a so-called thyroid index, which is a ratio LDH/CPK,
J5 a so-called estrogenic index, which is a ratio TSH/osteocalcin,
J6 a so-called growth index, which is a ratio bone isoenzymes of the alkaline phosphatases/osteocalcin, (O1/osteocalcin),
J7 a so-called turnover index, which is a product TSH×O1
J8 a so-called fibrosis index, J8 being defined by a relation
J8=(TSH)²(osteocalcin)³/100,
J9 a so-called index of thyroid involvement, which is a ratio CA 15-3/CEA,
J10 a so-called index of follicular involvement, which is a ratio CA 125/CEA,
J11 a so-called index of metabolic-hypothalamic involvement, which is the ratio CA19-9/CEA,
J12 a so-called pancreatic index, which is a ratio PAP/PSA,
J13 a so-called global TRH index of adaptation, which is a ratio CA19-9/TSH,
J14 a so-called index of leukocytes mobilization, J14 being defined by a relation J14=(platelets×neutrophils×HG)/(30×leukocytes),
J15 a so-called index of platelets mobilization, J15 being defined by a relation J15=platelets/(60×red blood cells),
J16 a so-called index of thyroid reactivating activity, which is a ratio monocytes/lymphocytes,
J17 a so-called structure/function ratio index, J17 being defined by a relation J17=(neutrophils+basophils+monocytes)/(eosinophils+lymphocytes),
J18 a so-called index of estrogenic fraction # 1, which is a ratio lymphocytes/osteocalcin,
J19 a so-called index of estrogenic fraction #2, which is a ratio neutrophils/monocytes,
J20 a so-called index of metabolic estrogenic fraction, which is a ratio LDH/osteocalcin,
J21 a so-called index of thyroid mobilization of bone metabolism, which is a ratio LDH/bone isoenzymes fraction of a alkaline phosphatases,
J22 a so-called index of thyroid mobilization of bone endocrine metabolism, which is a ratio TSH/bone isoenzymes fraction of a alkaline phosphatases,
J23 a so-called index of relative osteomuscular metabolic activity, which is a ratio CPK/bone isoenzymes fraction of a alkaline phosphatases,
J24 a so-called index of thyroid bone metabolic activity, which is a ratio CPK/osteocalcin,
J25 a so-called catabolism/anabolism ratio index, J25 being a ratio J2/J1,
J26 a so-called index of circulating cortisol, J26 being a ratio J25/J3,
J27 a so-called androgenic index, J27 being a ratio J1/J3,
J28 a so-called adrenal cortex index, J28 being a ratio J26/J27,
J29 a so-called index of adrenal cortex permissiveness, J29 being a ratio J1/J27,
J30 a so-called index of aromatization of estrogens, J30 being a ratio J29/J1,
J31 a so-called level of catabolism, J31 being a ratio J4/J28,
J32 a so-called level of anabolism, J32 being a ratio J31/J25,
J33 a so-called level of metabolic activity efficiency, J33 being defined by a relation J33=(J32+J31)×100/2.25,
J34 a so-called index of bone remodeling, which is a product TSH×J6,
J35 a so-called index of nuclear membrane activity, J35 being a ratio J5/J6,
J36 a so-called adjusted growth index, J36 being a ratio J6/J7,
J37 a so-called anti-growth index, J37 being a ratio 1/J36,
J38 a so-called somatostatin index, J38 being a ratio J37/J26,
J39 a so-called prolactin index, J39 being defined by a relation
J39=(J38/J6)×TSH,
J40 a so-called level of membrane expansion, J40 being a product J31×J36,
J41 a so-called level of structural expansion, J41 being a product J32×J35,
J42 a so-called apoptosis index, J42 being a ratio J41/J40,
J43 a so-called adjusted apoptosis index, J43 being a ratio J42/J35,
J44 a so-called level of membrane fracture, J44 being defined by a relation J44=J33/(TSH×J7),
J45 a so-called necrosis index, J45 being a ratio J44/J42,
J46 a so-called level of activity of total androgens, J46 being a product J5×J1
J47 a so-called rate of adrenal cortex androgens, J47 being defined by a relation J47=J46/(1+J27),
J48 a so-called rate of genital androgens, J48 being defined by a relation J48=(J46−J47),
J49 a so-called progesterone index, J49 being defined by a relation
J49=J5/(J48×J3),
J50 a so-called level of activity of genital estrogens, J50 being defined by a relation J50=J5/(1+J30),
J51 a so-called rate of aromatized estrogens, J51 being defined by a relation J51=J5−J50,
JJ53 a so-called folliculin index, J53 being defined by a relation J53=20×(J5/J49),
J54 a so-called insulin index, J54 being defined by a relation J54=(100×J25)/(J7×TSH),
J55 a so-called demyelinization index, J55 being defined by a relation J55−J541(J36×J6),
J56 a so-called index of DNA fracture, J56 being defined by a relation J56=(100×J5×J6×J41)/(J7×J35×J42×J45),
J57 a so-called index of nucleocytoplasmic pathogenicity, J57 being defined by a relation J57=(1.7×J56)/J44,
J58 a so-called index of cellular fracture, J58 being defined by a relation J58=2.5×J44×J56/J45,
J59 a so-called index of carcinogenesis, J59 is a ratio J57/J42,
J60 a so-called index of comparative carcinogenesis, J60 being defined by a relation J60=(10×J58)/J43,
J61 a so-called index of active cellular permeability, J61 being defined by a relation J61=J6×J34/J54,
J62 a so-called index of adjusted active cellular permeability, J62 being defined by a relation J62=(J61×J29)/J26,
J63 a so-called index of passive cellular permeability, J63 being defined by a relation J63=J45×J35×J68×10 (wherein J68 is defined as indicated below),
J64 a so-called index of active intracellular osmolar gradient, J64 being defined by a relation J64=100×J54×J40×J35/J3,
J65 a so-called index of adjusted active intracellular osmolar gradient, J65 being defined by a relation J65=(J64×J29)/J26,
J66 a so-called index of passive intracellular osmolar gradient, J66 being defined by a relation J66=(10×J43×J53)/(J45×J8),
J67 a so-called oxidation-reduction index, J67 being defined by a relation J67=(100×J45×J40×J41×J54)/(J71×J8×J38), (wherein J71 is defined as indicated below),
J68 a so-called index of corticoadrenal adaptation/permissiveness, J68 being defined by a relation J68=J26−J29−J28,
J69 a so-called adaptogenic index which is a ratio K/Ca,
J70 a so-called βMSH/αMSH index, (differential melanocyte-stimulating hormones), J70 being a ratio J4/J69,
J71 a so-called apoptosis bis index, J71 being defined by a relation J71=J35/(J36×J25),
J72 a so-called amylosis index, J72 being defined by a relation J72=(J38×J53×J55×TSH)/(J4×J5×J54),
J73 a so-called index of amylosis risk, J73 being a ratio J8/J67,
J74 a so-called index of insulin resistance, J74 being a ratio J38/J54,
J75 a so-called upstream index # 1, J75 being a ratio J4/J9,
J76 a so-called upstream index #2, J76 being a ratio J4/J10,
J77 a so-called upstream index #3, J77 being a ratio J4/J11,
J78 a so-called global upstream index #1, J78 being a ratio J75/J76,
J79 a so-called global upstream index #2, J79 being a ratio J75/J77,
J80 a so-called global upstream index #3, J80 being a ratio J76/J77,
J81 a so-called index of thyroid output #1, J81 being a ratio J4/TSH,
J82 a so-called index of free radicals, J82 being a ratio J67/J54,
J83 a so-called adjusted index of free radicals, J83 being defined by a relation J83=(J67+J64)/(J54+J74),
J84 a so-called comparative index of free radicals, J84 being defined by a relation J84=(J67+(100×J40)/(J54+J74),
J85 a so-called index of free radical nocivity, J85 being defined by a relation J85=((J82+J83+J84)×J56)/(3×J71),
J86 a so-called adjusted apoptosis index (B), J86 being a ratio J71/J35,
J87 a so-called index of active histamine, J87 being defined by a relation J87=(eosinophils×platelets×J3)/J52,
J88 a so-called index of potential histamine, J88 being defined by a relation J88=(J87×J63)/(potassium×J70)
J89 a so-called TRH index, J89 being a ratio TSH/FT4,
J90 a so-called index of relative intrathyroid TRH activity, which is a ratio FT3/FT4,
J91 a so-called index of carcinogenic expansion, J91 being a ratio J60/J59,
J92 a so-called index of cancer potential, J92 being a product
J91×J54×J85,
J93 a so-called adenosis index, J93 being a ratio J8/J91,
J94 a so-called ischemia reperfusion index, J94 being defined by a relation J94=10×J34×J43/J33,
J95 a so-called thrombogenic index, J95 being defined by a relation J95=10×J34×J42×J45/J33,
J96 a so-called thrombotic index, J96 being defined by a relation J96=J95×J87×J1/10,
J97 a so-called adjusted genital ratio index, J97 being defined by a relation J97=(J14×Red cells)/(Leukocytes×J15)=J14×J1/J15,
J98 a so-called musculotropic index, J98 being defined by a relation J98=J97×(CPK/O1),
J99 a so-called adjusted estrogenic index, J99 being defined by a relation J99=(J5×(osteocalcin+1)/(osteocalcin+1−J98)),
J100 a so-called genital androgeny index, J100 being defined by a relation J100=(J98/J81)×J99×(J97)²/(J3+J97),
J101 a so-called comparative genital androgeny index, J101 being defined by a relation J101=(2×(TSH)²×CPK)/(J4×osteocalcin×01),
J102 a so-called “starter” index, J102 being a ratio J14/J15,
J103 a so-called adjusted index of thyroid reactivating activity, J103 being a product J16×J2,
J104 a so-called pro-inflammatory index, J104 being a product J103×J69,
J105 a so-called index of inflammation, J105 being a product J104×J45,
J106 a so-called comparative index of inflammation, J106 being defined by a relation J106=J105/(((ESR₂/2)+ESR₁)/2)/ESR₁,
J107 a so-called interleukin 1 index, J107 being defined by a relation J107=(J16×J38)/(J103×J37),
J108 a so-called DHEA index, J108 being defined by a relation J108=(J29×J30×J47×J51×J98×1000)/(J49×J27×J100),
J109 a so-called serotonin index, J109 being defined by a relation J109=(10×J102)/(J54×J74),
J110 a so-called adjusted demyelinization index, J110 being a product J55×J102,
J111 a so-called expansiveness index #1, J111 being a ratio J36/J35,
J112 a so-called expansiveness index #2, J112 being a ratio J40/J41,
J113 a so-called global expansiveness index, J113 being defined by a relation J113=(J111×J112)/J45,
J114 a so-called ACTH index, J114 being a ratio J108/J26,
J115 a so-called PTH index, J115 being defined by a relation J115=(calcium×osteocalcin×TSH)/J4,
J116 a so-called index of gonadotropic output, J116 being defined by a relation J116=1/(J×J53),
J117 a so-called index of pelvic congestion, J117 being defined by a relation J117=(J59/J60)×(J94/J33),
J118 a so-called index of splanchnic congestion, J118 being a ratio J117/J14,
J119 a so-called growth score index, J119 being defined by a relation J119=(J6×J37)/(J36×J38),
J120 a so-called GH growth score index, J120 being defined by a relation J120=(J6×J37)/J36,
J121 a so-called TRH/TSH ratio index, J121 being a ratio J72/J93,
J122 a so-called index of thyroid efficiency, J122 being a ratio J4/J2,
J123 a so-called index of relative thyroid efficiency, J123 being a ratio J122/J81,
J124 a so-called index of oxidation, J124 being defined by a relation J124=(100×J36×J54×J122)/(J74×J26),
J125 a so-called index of reduction, J125 being a ratio J124/J67,
J126 a so-called pro-amyloid index, J126 being a product J125×J74,
J127 a so-called index of amyloid risk, J127 being a ratio J8/J124,
J128 a so-called index of thyroid output #2, J128 being a product J2×J4,
J129 a so-called comparative index of thyroid output, J129 being a ratio J128/J81,
J130 a so-called index of estrogenic fraction #3, J130 being a ratio 1/J1,
J131 a so-called index of estrogenic fraction #4, J131 being a product J18×J19,
J132 a so-called index of estrogenic fraction #5, J132 being a product J19×J130,
J133 a so-called general index of estrogenic fraction, J133 being a product J18×J19×J130,
J134 a so-called index of estrogenic fraction #6, J134 being defined by a relation J134=1/(osteocalcin×J2),
J135 a so-called index of estrogenic fraction #7, J135 being a product J18×J19×J134,
J136 a so-called index of estrogenic fraction #8, J136 being a ratio J2/osteocalcin,
J137 a so-called general quantitative estrogenic index, J137 being a product (J18+J19)×(leukocytes/100),
J138 a so-called index of specific estrogenic fraction, J138 being a product J5×(J98+1),
J139 a so-called comparative estrogenic index #1, J139 being a ratio J133/(J5×100),
J140 a so-called comparative estrogenic index #2, J140 being a ratio J133/(J99×100),
J141 a so-called global comparative estrogenic index, J141 being a ratio J133/(J5×J99×100),
J142 a so-called index of somatotropic estrogenic output, J142 being a ratio J133/J144 (where J144 is defined as indicated below),
J143 a so-called index of quantitative organotissular estrogenic output, J143 being a ratio J137/J144 (where J144 is defined below),
J144 a so-called FSH index #1, J144 being a ratio J114/J3,
J145 a so-called LH index #1, J145 being a product J114×J27,
J146 a so-called FSH index #2, J146 being a ratio J145/J1,
J147 a so-called LH index #2, J147 being a product J144×J1,
J148 a so-called index of progesterone output, J148 being a ratio J49/J138,
J149 a so-called ketonic index, J149 being a ratio J102/J54,
J150 a so-called index of total subliminal TRH, J150 being a product TSH×(CA19-9)×J90,
J151 a so-called index of active carcinogenesis, J151 being a product J59×J113,
J152 a so-called comparative index of active carcinogenesis, J152 being a product J60×J113,
J153 a so-called gonadothyrotropic index, J153 being a ratio TSH/J2,
J154 a so-called index of global tissular estrogenic fraction, J154 being a ratio J140/J139,
J155 a so-called index of muscle destruction, J155 being a ratio J36/J101,
J156 a so-called amyloid score index, J156 being defined by a relation J156=(J2×J53×J72×J94×J110×J126×J127)/(J4×J5×J67×J19×J20),
J157 a so-called adjusted necrosis index, J157 being a product LDH×J45; and, (4°) comparing at least one of the J1-J157 indexes with the corresponding result obtained according to steps (2°) and (3°) with human beings already recognized as being healthy, in order to appreciate dynamically the biological state of the patient to be tested.

2. A method according to claim 1, wherein at least one abnormality is looked for at the level of indexes J1 to J157 of the patient.

3. A method according to claim 1, wherein (i) at least one index from J1 to J24 and (ii) at least one index from J25 to J157 are looked for in step (3°).

4. A method according to claim 1, wherein in step (3°) at least 8, 10 or 15 indexes J are measured.

5. A method according to claim 1, wherein in step (3°) at least a portion of indexes J1-J157, which constitute the scores of functions involved in at least one of symptomatology and/or pathology of the patient to be tested, is measured.

6. A method according to claim 5, wherein said scores of functions involved in the symptomatology and/or pathology are selected from the group consisting of the scores of pregnancy, menstruation, cardiovascularity, thrombosis, Alzheimer's disease, atherosclerosis, cancer and sudden death risk.

7. A method according to claim 1, wherein in step (4°) the indexes obtained for a patient to be tested are compared with corresponding median values determined from healthy subjects.

8. A software product loadable in particular in an internal memory of a computer, comprising portions of computer code to perform the following steps:
(3°) measuring, from step (2°), at least one index selected from the group consisting of following indexes J1-J157:
J1 a so-called genital ratio index, which is a ratio red blood cells/leukocytes,
J2 a so-called genital-thyroid ratio index, which is a ratio neutrophils/lymphocytes,
J3 a so-called adaptation index, which is a ratio eosinophils/monocytes, J3 being such that J3=eosinophils/monocytes=ACTH/FSH,
J4 a so-called thyroid index, which is a ratio LDH/CPK,
J5 a so-called estrogenic index, which is a ratio TSH/osteocalcin,
J6 a so-called growth index, which is a ratio bone isoenzymes of the alkaline phosphatases/osteocalcin, (O1/osteocalcin),
J7 a so-called turnover index, which is a product TSH×O1
J8 a so-called fibrosis index, J8 being defined by a relation J8=(TSH)²(osteocalcin)³/100,
J9 a so-called index of thyroid involvement, which is a ratio CA 15-3/CEA,
J10 a so-called index of follicular involvement, which is a ratio CA 125/CEA,
J11 a so-called index of metabolic-hypothalamic involvement, which is a ratio CA19-9/CEA,
J12 a so-called pancreatic index, which is a ratio PAP/PSA,
J13 a so-called global TRH index of adaptation, which is a ratio CA19-9/TSH,
J14 a so-called index of leukocytes mobilization, J14 being defined by a relation J14=(platelets×neutrophils×HG)/(30×leukocytes),
J15 a so-called index of platelets mobilization, J15 being defined by a relation J15=platelets/(60×red blood cells),
J16 a so-called index of thyroid reactivating activity, which is a ratio monocytes/lymphocytes,
J17 a so-called structure/function ratio index, J17 being defined by a relation J17=(neutrophils+basophils+monocytes)/(eosinophils+lymphocytes),
J18 a so-called index of estrogenic fraction # 1, which is a ratio lymphocytes/osteocalcin,
J19 a so-called index of estrogenic fraction #2, which is a ratio neutrophils/monocytes,
J20 a so-called index of metabolic estrogenic fraction, which is a ratio LDH/osteocalcin,
J21 a so-called index of thyroid mobilization of bone metabolism, which is a ratio LDH/bone isoenzymes fraction of a alkaline phosphatases,
J22 a so-called index of thyroid mobilization of bone endocrine metabolism, which is a ratio TSH/bone isoenzymes fraction of a alkaline phosphatases,
J23 a so-called index of relative osteomuscular metabolic activity, which is a ratio CPK/bone isoenzymes fraction of a alkaline phosphatases,
J24 a so-called index of thyroid bone metabolic activity, which is a ratio CPK/osteocalcin,
J25 a so-called catabolism/anabolism ratio index, J25 being a ratio J2/J1,
J26 a so-called index of circulating cortisol, J26 being a ratio J25/J3,
J27 a so-called androgenic index, J27 being a ratio J1/J3,
J28 a so-called adrenal cortex index, J28 being a ratio J26/J27,
J29 a so-called index of adrenal cortex permissiveness, J29 being a ratio J1/J27,
J30 a so-called index of aromatization of estrogens, J30 being a ratio J29/J1,
J31 a so-called level of catabolism, J31 being a ratio J4/J28,
J32 a so-called level of anabolism, J32 being a ratio J31/J25,
J33 a so-called level of metabolic activity efficiency, J33 being defined by a relation J33=(J32+J31)×100/2.25,
J34 a so-called index of bone remodeling, which is a product TSH×J6,
J35 a so-called index of nuclear membrane activity, J35 being a ratio J5/J6,
J36 a so-called adjusted growth index, J36 being a ratio J6/J7,
J37 a so-called anti-growth index, J37 being a ratio 1/J36,
J38 a so-called somatostatin index, J38 being a ratio J37/J26,
J39 a so-called prolactin index, J39 being defined by a relation J39=(J38/J6)×TSH,
J40 a so-called level of membrane expansion, J40 being a product J31×J36,
J41 a so-called level of structural expansion, J41 being a product J32×J35,
J42 a so-called apoptosis index, J42 being a ratio J41/J40,
J43 a so-called adjusted apoptosis index, J43 being a ratio J42/J35,
J44 a so-called level of membrane fracture, J44 being defined by a relation J44=J33/(TSH×J7),
J45 a so-called necrosis index, J45 being a ratio J44/J42,
J46 a so-called level of activity of total androgens, J46 being a product J5×J1
J47 a so-called rate of adrenal cortex androgens, J47 being defined by a relation J47=J46/(1+J27),
J48 a so-called rate of genital androgens, J48 being defined by a relation J48=(J46−J47),
J49 a so-called progesterone index, J49 being defined by a relation
J49=J5/(J48×J3),
J50 a so-called level of activity of genital estrogens, J50 being defined by a relation J50=J5/(1+J30),
J51 a so-called rate of aromatized estrogens, J51 being defined by a relation J51=J5−J50,
JJ53 a so-called folliculin index, J53 being defined by a relation J53=20×(J5/J49),
J54 a so-called insulin index, J54 being defined by a relation J54=(100×J25)/(J7×TSH),
J55 a so-called demyelinization index, J55 being defined by a relation J55−J541(J36×J6),
J56 a so-called index of DNA fracture, J56 being defined by a relation J56=(100×J5×J6×J41)/(J7×J35×J42×J45),
J57 a so-called index of nucleocytoplasmic pathogenicity, J57 being defined by a relation J57=(1.7×J56)/J44,
J58 a so-called index of cellular fracture, J58 being defined by a relation J58=2.5×J44×J56/J45,
J59 a so-called index of carcinogenesis, J59 is a ratio J57/J42,
J60 a so-called index of comparative carcinogenesis, J60 being defined by a relation J60=(10×J58)/J43,
J61 a so-called index of active cellular permeability, J61 being defined by a relation J61=J6×J34/J54,
J62 a so-called index of adjusted active cellular permeability, J62 being defined by a relation J62=(J61×J29)/J26,
J63 a so-called index of passive cellular permeability, J63 being defined by a relation J63=J45×J35×J68×10 (wherein J68 is defined as indicated below),
J64 a so-called index of active intracellular osmolar gradient, J64 being defined by a relation J64=100×J54×J40×J35/J3,
J65 a so-called index of adjusted active intracellular osmolar gradient, J65 being defined by a relation J65=(J64×J29)/J26,
J66 a so-called index of passive intracellular osmolar gradient, J66 being defined by a relation J66=(10×J43×J53)/(J45×J8),
J67 a so-called oxidation-reduction index, J67 being defined by a relation J67=(100×J45×J40×J41×J54)/(J71×J8×J38), (wherein J71 is defined as indicated below),
J68 a so-called index of corticoadrenal adaptation/permissiveness, J68 being defined by a relation J68=J26−J29−J28,
J69 a so-called adaptogenic index which is a ratio K/Ca,
J70 a so-called βMSH/αMSH index, (differential melanocyte-stimulating hormones), J70 being a ratio J4/J69,
J71 a so-called apoptosis bis index, J71 being defined by a relation J71=J35/(J36×J25),
J72 a so-called amylosis index, J72 being defined by a relation J72=(J38×J53×J55×TSH)/(J4×J5×J54),
J73 a so-called index of amylosis risk, J73 being a ratio J8/J67,
J74 a so-called index of insulin resistance, J74 being a ratio J38/J54,
J75 a so-called upstream index # 1, J75 being a ratio J4/J9,
J76 a so-called upstream index #2, J76 being a ratio J4/J10,
J77 a so-called upstream index #3, J77 being a ratio J4/J11,
J78 a so-called global upstream index #1, J78 being a ratio J75/J76,
J79 a so-called global upstream index #2, J79 being a ratio J75/J77,
J80 a so-called global upstream index #3, J80 being a ratio J76/J77,
J81 a so-called index of thyroid output #1, J81 being a ratio J4/TSH,
J82 a so-called index of free radicals, J82 being a ratio J67/J54,
J83 a so-called adjusted index of free radicals, J83 being defined by a relation J83=(J67+J64)/(J54+J74),
J84 a so-called comparative index of free radicals, J84 being defined by a relation J84=(J67+(100×J40)/(J54+J74),
J85 a so-called index of free radical nocivity, J85 being defined by a relation J85=((J82+J83+J84)×J56)/(3×J71),
J86 a so-called adjusted apoptosis index (B), J86 being a ratio J71/J35,
J87 a so-called index of active histamine, J87 being defined by a relation
J87=(eosinophils×platelets×J3)/J52,
J88 a so-called index of potential histamine, J88 being defined by a relation J88=(J87×J63)/(potassium×J70)
J89 a so-called TRH index, J89 being a ratio TSH/FT4,
J90 a so-called index of relative intrathyroid TRH activity, which is a ratio FT3/FT4,
J91 a so-called index of carcinogenic expansion, J91 being a ratio J60/J59,
J92 a so-called index of cancer potential, J92 being a product
J91×J54×J85,
J93 a so-called adenosis index, J93 being a ratio J8/J91,
J94 a so-called ischemia reperfusion index, J94 being defined by a relation J94=10×J34×J43/J33,
J95 a so-called thrombogenic index, J95 being defined by a relation J95=10×J34×J42×J45/J33,
J96 a so-called thrombotic index, J96 being defined by a relation J96=J95×J87×J1/10,
J97 a so-called adjusted genital ratio index, J97 being defined by a relation J97=(J14×Red cells)/(Leukocytes×J15)=J14×J1/J15,
J98 a so-called musculotropic index, J98 being defined by a relation J98=J97×(CPK/O1),
J99 a so-called adjusted estrogenic index, J99 being defined by a relation J99=(J5×(osteocalcin+1)/(osteocalcin+1−J98)),
J100 a so-called genital androgeny index, J100 being defined by a relation J100=(J98/J81)×J99×(J97)²/(J3+J97),
J101 a so-called comparative genital androgeny index, J101 being defined by a relation J101=(2×(TSH)²×CPK)/(J4×osteocalcin×01),
J102 a so-called “starter” index, J102 being a ratio J14/J15,
J103 a so-called adjusted index of thyroid reactivating activity, J103 being a product J16×J2,
J104 a so-called pro-inflammatory index, J104 being a product J103×J69,
J105 a so-called index of inflammation, J105 being a product J104×J45,
J106 a so-called comparative index of inflammation, J106 being defined by a relation J106=J105/(((ESR₂/2)+ESR₁)/2)/ESR₁,
J107 a so-called interleukin 1 index, J107 being defined by a relation J107=(J16×J38)/(J103×J37),
J108 a so-called DHEA index, J108 being defined by a relation J108=(J29×J30×J47×J51×J98×1000)/(J49×J27×J100),
J109 a so-called serotonin index, J109 being defined by a relation J109=(10×J102)/(J54×J74),
J110 a so-called adjusted demyelinization index, J110 being a product J55×J102,
J111 a so-called expansiveness index #1, J111 being a ratio J36/J35,
J112 a so-called expansiveness index #2, J112 being a ratio J40/J41,
J113 a so-called global expansiveness index, J113 being defined by a relation J113=(J111×J112)/J45,
J114 a so-called ACTH index, J114 being a ratio J108/J26,
J115 a so-called PTH index, J115 being defined by a relation J115=(calcium×osteocalcin×TSH)/J4,
J116 a so-called index of gonadotropic output, J116 being defined by a relation J116=1/(J×J53),
J117 a so-called index of pelvic congestion, J117 being defined by a relation J117=(J59/J60)×(J94/J33),
J118 a so-called index of splanchnic congestion, J118 being a ratio J117/J14,
J119 a so-called growth score index, J119 being defined by a relation J119=(J6×J37)/(J36×J38),
J120 a so-called GH growth score index, J120 being defined by a relation J120=(J6×J37)/J36,
J121 a so-called TRH/TSH ratio index, J121 being a ratio J72/J93,
J122 a so-called index of thyroid efficiency, J122 being a ratio J4/J2,
J123 a so-called index of relative thyroid efficiency, J123 being a ratio J122/J81,
J124 a so-called index of oxidation, J124 being defined by a relation J124=(100×J36×J54×J122)/(J74×J26),
J125 a so-called index of reduction, J125 being a ratio J124/J67,
J126 a so-called pro-amyloid index, J126 being a product J125×J74,
J127 a so-called index of amyloid risk, J127 being a ratio J8/J124,
J128 a so-called index of thyroid output #2, J128 being a product J2×J4,
J129 a so-called comparative index of thyroid output, J129 being a ratio J128/J81,
J130 a so-called index of estrogenic fraction #3, J130 being a ratio 1/J1,
J131 a so-called index of estrogenic fraction #4, J131 being a product J18×J19,
J132 a so-called index of estrogenic fraction #5, J132 being a product J19×J130,
J133 a so-called general index of estrogenic fraction, J133 being a product J18×J19×J130,
J134 a so-called index of estrogenic fraction #6, J134 being defined by a relation J134=1/(osteocalcin×J2),
J135 a so-called index of estrogenic fraction #7, J135 being a product J18×J19×J134,
J136 a so-called index of estrogenic fraction #8, J136 being a ratio J2/osteocalcin,
J137 a so-called general quantitative estrogenic index, J137 being a product (J18+J19)×(leukocytes/100),
J138 a so-called index of specific estrogenic fraction, J138 being a product J5×(J98+1),
J139 a so-called comparative estrogenic index #1, J139 being a ratio J133/(J5×100),
J140 a so-called comparative estrogenic index #2, J140 being a ratio J133/(J99×100),
J141 a so-called global comparative estrogenic index, J141 being a ratio J133/(J5×J99×100),
J142 a so-called index of somatotropic estrogenic output, J142 being a ratio J133/J144 (where J144 is defined as indicated below),
J143 a so-called index of quantitative organotissular estrogenic output, J143 being a ratio J137/J144 (where J144 is defined below),
J144 a so-called FSH index #1, J144 being a ratio J114/J3,
J145 a so-called LH index #1, J145 being a product J114×J27,
J146 a so-called FSH index #2, J146 being a ratio J145/J1,
J147 a so-called LH index #2, J147 being a product J144×J1,
J148 a so-called index of progesterone output, J148 being a ratio J49/J138,
J149 a so-called ketonic index, J149 being a ratio J102/J54,
J150 a so-called index of total subliminal TRH, J150 being a product TSH×(CA19-9)×J90,
J151 a so-called index of active carcinogenesis, J151 being a product J59×J113,
J152 a so-called comparative index of active carcinogenesis, J152 being a product J60×J113,
J153 a so-called gonadothyrotropic index, J153 being a ratio TSH/J2,
J154 a so-called index of global tissular estrogenic fraction, J154 being a ratio J140/J139,
J155 a so-called index of muscle destruction, J155 being a ratio J36/J101,
J156 a so-called amyloid score index, J156 being defined by a relation J156=(J2×J53×J72×J94×J110×J126×J127)/(J4×J5×J67×J19×J20),
J157 a so-called adjusted necrosis index, J157 being a product LDH×J45; and,
(4°) comparing at least one of the J1-J157 indexes with the corresponding result obtained according to steps (2°) and (3°) with human beings already recognized as being healthy, in order to appreciate dynamically the biological state of the patient to be tested.

9. A software product according to claim 8, comprising
(A) a form field for entering (i) the patient's name or code, (ii) his age, and (iii) his sex, on the one hand, and any known treatment followed by said patient formerly or at the present time, on the other hand;
(B) a form field for entering (iv) one or several hematic parameters measured in vitro from the blood of the patient and (v) the date of the measures;
(C) a field including all the indexes from J1 to J157, (vii) their calculation mode and (viii) their median values determined from human beings who are recognized as healthy subjects;
(D) a command for (ix) the calculation of one index J, of several indexes J or the totality of indexes from the hematic parameters of said field (B) obtained from the blood of the patient to be tested, and (x) for the comparison of the obtained value for at least one of said indexes J with its median value of field (C); and,
(E) means for classifying, visualizing, editing and/or printing the obtained result by implementing command (D) starting from fields (A), (B), and (C).

10. A software product according to claim 9, wherein each median value of an index J in an interval of normal values previously determined from subjects recognized as being healthy.

11. A software product according to claim 10, which comprises an instruction for recognizing and flagging up any abnormality constituted by a value of an index J which stands away from its median value or interval thereof.

12. A software product according to claim 9, which comprises an instruction for recognizing and flagging up any abnormality constituted by a value of an index J which stands away from its median value or interval thereof.
